(19) United States
(12) Patent Application Publication
(10) Pub. No.: US 2020/0215233 A1
(43) Pub. Date: Jul. 9, 2020

(54) ANTIMICROBIAL COATING

(71) Applicant: Boulos & Cooper Pharmaceuticals Pty Ltd, Balcatta (AU)

(72) Inventors: Ramiz Boulos, Yokine Western Australia (AU); Berkay Ozcelik, Dingley Village Victoria (AU); Helmut Thissen, Rowville Victoria (AU)

(21) Appl. No.: 16/640,758

(22) PCT Filed: Aug. 22, 2018
(86) PCT No.: PCT/AU2018/050891

§ 371 Date: Feb. 21, 2020

(30) Foreign Application Priority Data
Aug. 24, 2017 (AU) 2017903422

(51) Int. Cl. A61L 29/16; A61L 29/08; A61L 29/14

(52) U.S. Cl. CPC A61L 29/16; A61L 29/085; C08L 67/04; A61L 2420/06; A61L 29/148

(57) ABSTRACT

The invention disclosed herein relates to compositions for coating a surface to provide a release of antimicrobial active agents from said surface, to coatings formed using said compositions, and to medical devices comprising said coatings.

### TECHNICAL FIELD

The present invention relates to a composition for coating a surface to provide a release of antimicrobial active agents, to coatings formed using said compositions, and to medical devices comprising said coatings.

### BACKGROUND ART

Advances in medical device technologies have contributed significantly to the increase in life expectancy and, perhaps more importantly, the quality of life for an aging population. However, some challenges associated with the application of these devices remain, including medical device associated infections. Although the frequency of infection over the lifetime of the device varies significantly, the risk of an infection exists for all medical devices. In most cases, the infection is established by microbial colonization of the device, followed by the formation of a biofilm, a dense matrix of proteins, polysaccharides and DNA in which the bacteria become embedded.

To address this challenge, multiple approaches have been developed for the application of antimicrobial coatings to medical devices. However, many devices continue to be used without any additional protection to reduce the risk of infection. In addition, many of the causative organisms have become increasingly resistant to commonly used antimicrobial agents and new antimicrobial compounds with new drug targets are urgently required.

The mechanism of release is the rate limiting step or series of rate limiting steps that control the rate of drug release from a device such as an antimicrobially coated medical device until release is exhausted. The major release mechanisms include: diffusion, solvent penetration/device swelling, degradation and erosion of the polymer matrix, or a combination of these mechanisms occurring on different time scales that leads to a more complex release process. Further discussion of this is provided in Hines and Kaplan (2013) Crit Rev Ther Drug Carrier Syst. 30(3): 257-276.

The most desirable case is zero order release kinetics. In such a case the rate of drug release is independent of its dissolved concentration in the release medium and is delivered at a constant rate over time. This type of release is unachievable by current polymeric release systems. Diffusion is the most common release mechanism and is dependent on the concentration of the dissolved drug as described by Fick's second Law. The rate of release for diffusion has a half ordered time dependency. Erodible delivery systems are also non-zero ordered and the rate of release is dependent on the degradation kinetics of the polymer used. Solvent penetration systems are also non-zero ordered and their rate is dependent upon the permeability of the polymer used. Typically the polymer and the processing modes selected for the device formulation influence the mechanism of release. Internal diffusion to the surface of the delivery device is the most common release mechanism.

Ventilator-associated pneumonia (VAP) is one of the most common nosocomial infections that occurs in people who require intubation and mechanical ventilation. VAP typically affects people in intensive care units and represents a major unresolved clinical problem. For patients that contract VAP, the result can be an increased length of hospitalization or death (at a rate of 20-30%). Endotracheal tubes (ETTs) that are utilized during ventilation are considered to be a major risk factor for VAP. The endotracheal tube provides a surface that allows colonization and biofilm formation by bacteria including Staphylococcus aureus. In order to reduce the incidence of VAP associated with ETT use, various methods including prevention of aspiration of secretions, antimicrobial rinsing, photodynamic therapies and antimicrobial releasing coatings have been explored. Particularly regarding antimicrobial coatings for ETTs, silver releasing coatings have been the most widely studied.

There is a need to provide improved or alternative coatings for medical devices; or at least the provision of alternative coatings to compliment the previously known coating methods.

The above discussion of the background art is intended to facilitate an understanding of the present invention only. The discussion is not an acknowledgement or admission that any of the material referred to is or was part of the common general knowledge as at the priority date of the application.

### SUMMARY OF INVENTION

In one aspect of the present invention, there is provided a composition for coating a surface, said composition comprising;
a. a polymer selected from the group: poly(lactic-co-glycolic acid), polylactide, polyglycolide, polyester, polyurethane and combinations thereof; and
b. an antimicrobial compound of Formula (I);
wherein the compound of Formula (I) is a compound having a structure selected from Group I, wherein Group I consists of;

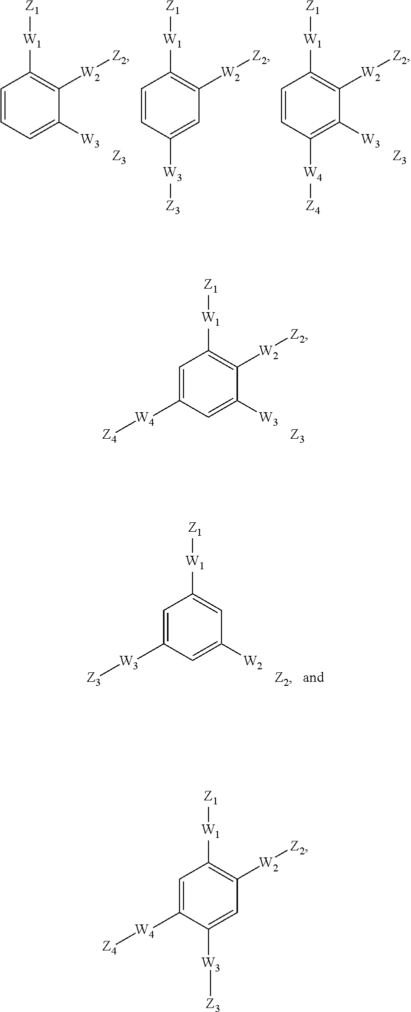

wherein
each of W₁, W₂, W₃, and W₄ is the same and is selected from the group consisting of C₂₋₄ alkyl, substituted C₂₋₄ alkyl; and C₂ alkene;
each of Z₁, Z₂, Z₃, and Z₄ is the same and each is selected from the group consisting of;

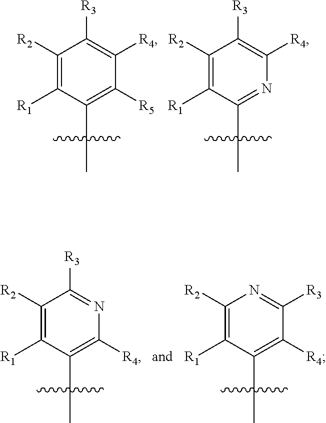

each of R₁, R₂, R₃, R₄, and R₅ is independently H or C₁₋₈ heteroalkyl, wherein the C₁₋₈ heteroalkyl comprises —CO₂H or an ester thereof, with the proviso that at least one of R₁, R₂, R₃, R₄, and R₅ is C₁₋₈ heteroalkyl, or a pharmaceutically acceptable salt thereof.

In one form of the present invention, the composition provides a self-limiting drug release profile of the compound of Formula (I).

In one form of the present invention, the polymer of the composition for coating a surface, is a biodegradable polymer.

In one aspect of the present invention, the C₁ heteroalkyl group of Formula (I) is —CO₂H or an ester thereof.

In one form of the invention, the composition provides a self-limiting drug release profile, via concentration dependent release of an antibiotic compound of Formula (I).

In one aspect of the present invention, there is provided a composition for coating a surface, wherein the composition comprises;
a. a polymer selected from the group: poly(lactic-co-glycolic acid), polylactide, polyglycolide, polyester, polyurethane and combinations thereof; and
b. an antimicrobial compound of Formula (I);
wherein the concentration of the antimicrobial compound of Formula (I) in the composition is at least 0.01 mg/mL.

In one aspect of the present invention, there is provided a coating, wherein the coating comprises;
a. a polymer selected from the group: poly(lactic-co-glycolic acid), polylactide, polyglycolide, polyester, polyurethane and combinations thereof; and
b. an antimicrobial compound of Formula (I);
   wherein the amount of the antimicrobial compound of Formula (I) per unit surface of the coating is at least 0.2 μg/cm2.

In one aspect of the present invention, there is provided a coating, wherein the coating comprises;
a. a polymer selected from the group: poly(lactic-co-glycolic acid), polylactide, polyglycolide, polyester, polyurethane and combinations thereof; and
b. an antimicrobial compound of Formula (I);
   and wherein, when a sample of the coating is placed into a well of tissue culture polystyrene (TOPS) 48-well plate and 500 μL of fresh 1× phosphate-buffered saline is pipetted into each well containing the sample of coating such that complete coverage of each sample of the coating with the phosphate-buffered saline is ensured, and the well of tissue culture polystyrene is sealed and placed in a shaker incubator at 37° C. (80 rpm), the concentration of the antimicrobial compound of formula (I) in the solution at 24 hours is at least 0.05 μg of the compound of Formula (I) per cm2 of coating surface per mL.

In embodiments of the aforementioned coatings the concentration of the antimicrobial compound of Formula (I) at the surface to which the coating is applied is an effective inhibitory concentration for bacteria selected from the group: gram positive bacteria and gram negative bacteria and combinations thereof.

In embodiments of the aforementioned coatings the concentration of the antimicrobial compound of Formula (I) at the surface to which the coating is applied is an effective inhibitory concentration for gram positive bacteria.

In embodiments of the aforementioned coatings the concentration of the antimicrobial compound of Formula (I) at the surface to which the coating is applied is an effective inhibitory concentration for gram positive bacteria wherein the gram positive bacteria is S. aureus.

In embodiments of the aforementioned coatings the concentration of the antimicrobial compound of Formula (I) at the surface to which the coating is applied is an effective inhibitory concentration for gram negative bacteria.

In embodiments of the aforementioned coatings the concentration of the antimicrobial compound of Formula (I) at the surface to which the coating is applied is an effective inhibitory concentration for gram negative bacteria wherein the gram negative bacteria is P. aeruginosa.

In embodiments of the aforementioned coatings the concentration of the antimicrobial compound of Formula (I) at the surface to which the coating is applied is an effective inhibitory concentration for bacteria selected from the group: S. aureus and P. aeruginosa and combinations thereof.

In embodiments of the aforementioned coatings the concentration of the antimicrobial compound of Formula (I) at the surface to which the coating is applied is a useful inhibitory concentration for bacteria selected from the group: gram positive bacteria and gram negative bacteria and combinations thereof.

In embodiments of the aforementioned coatings the concentration of the antimicrobial compound of Formula (I) at the surface to which the coating is applied is a useful inhibitory concentration for gram positive bacteria.

In embodiments of the aforementioned coatings the concentration of the antimicrobial compound of Formula (I) at the surface to which the coating is applied is a useful inhibitory concentration for gram positive bacteria wherein the gram positive bacteria is S. aureus.

In embodiments of the aforementioned coatings the concentration of the antimicrobial compound of Formula (I) at the surface to which the coating is applied is a useful inhibitory concentration for gram negative bacteria.

In embodiments of the aforementioned coatings the concentration of the antimicrobial compound of Formula (I) at the surface to which the coating is applied is a useful inhibitory concentration for gram negative bacteria wherein the gram negative bacteria is P. aeruginosa.

In embodiments of the aforementioned coatings the concentration of the antimicrobial compound of Formula (I) at the surface to which the coating is applied is a useful inhibitory concentration for bacteria selected from the group: S. aureus and P. aeruginosa and combinations thereof.

In one aspect of the present invention, there is provided a coating, wherein the coating comprises:
a. a polymer selected from the group: poly(lactic-co-glycolic acid), polylactide, polyglycolide, polyester, polyurethane and combinations thereof; and
b. an antimicrobial compound of Formula (I);
   wherein, when the coating is placed in an aqueous solution, and wherein, when a sample of the coating is placed into a well of tissue culture polystyrene (TOPS) 48-well plate and 500 μL of fresh 1× phosphate-buffered saline is pipetted into each well containing the sample of coating such that complete coverage of each sample of the coating with the phosphate-buffered saline is ensured, and the well of tissue culture polystyrene is sealed and placed in a shaker incubator at 37° C. (80 rpm), and wherein at 1, 4, 24 and 48 h, 100 μL of solution was removed, and wherein at 72 h and at 8 and 24 days all 500 μL of solution was removed and replaced with 500 μL of fresh 1× phosphate-buffered saline, the concentration of antimicrobial compound in the aqueous solution after 31 days is at least 5 μg of the compound of Formula (I) per cm² of coating surface per mL.

In one aspect of the present invention there is provided a coating according to any one of the aforementioned embodiments wherein the thickness of the coating is at least 4 μm and the concentration of the compound of Formula I is at least 25 μg/cm².

In one aspect of the present invention there is provided a coating or composition according to any one of the embodiments characterised in that the surface forms part of a medical device.

In one aspect of the present invention there is provided a coating or composition according to any one of the embodiments characterised in that the medical device is an endotracheal tube.

In a preferred form of any of the aforementioned aspects or embodiments of the invention, the polymer is poly(lactic-co-glycolic acid).

In one embodiment of the present invention there is provided a coating or composition according to any one of the aforementioned aspects or embodiments characterised in the antimicrobial compound of Formula (I) is a compound of Formula E, or a pharmaceutically acceptable salt thereof.

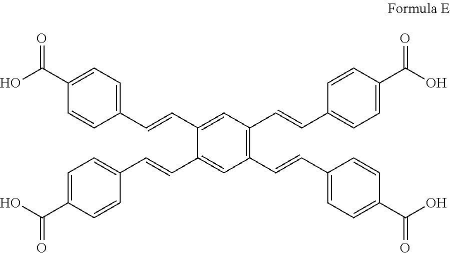 [SMILES: O=C(O)c1ccc(/C=C/c2cc(/C=C/c3ccc(C(=O)O)cc3)c(/C=C/c3ccc(C(=O)O)cc3)cc2/C=C/c2ccc(C(=O)O)cc2)cc1]

In a highly preferred form of any of the aforementioned aspects or embodiments of the invention, the antimicrobial compound of Formula (I) is a compound of Formula E, or a pharmaceutically acceptable salt thereof and the polymer is poly(lactic-co-glycolic acid), also referred to herein as PLGA.

### BRIEF DESCRIPTION OF THE DRAWINGS

The description will be made with reference to the accompanying drawings in which:

FIG. 1 is the structure of Formula E, also referred to herein as BCP3, a representative antimicrobial for use in the present invention.

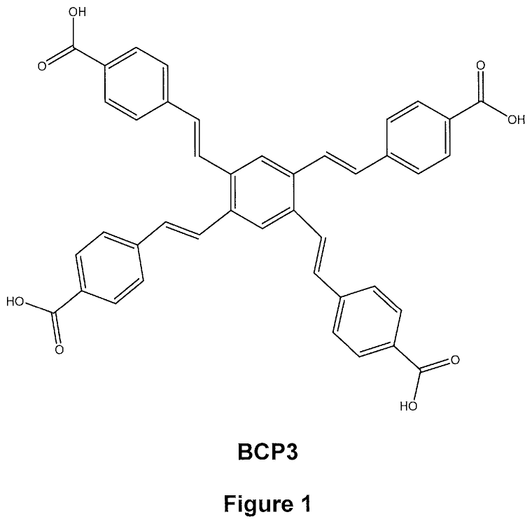

FIG. 2 is a schematic of the coating process.

FIG. 3 is a range of SEM cross-section images of some of the coated ETTs showing a distinct coating presence.

FIGS. 4A and 4B are graphs showing 4A) coating thickness at different PLGA and BCP3 concentrations, 4B) BCP3 loading (mg/cm²) on the ETT segments at each coating formulation.

FIGS. 5A and 5B are graphs showing release of BCP3 from the coatings over 5A) 31 days and 5B) 72 h. A concentration dependent release can be observed for at least 31 days. After 48 h, the ETT segments were incubated in completely fresh phosphate buffered saline (PBS).

FIGS. 6A and 6B are graphs showing in vitro 6A) methicillin sensitive S. aureus and 6B) methicillin resistant S. aureus bacterial inhibition assays of various coating formulations.

FIG. 7 is a graph showing P. aeruginosa bacterial inhibition assay of various coating formulations.

FIGS. 8A and 8B are graphs showing in vitro cell viability in the presence of 8A) BCP3 up to 1 mg/mL concentration (concentrations below 0.125 mg/mL not shown), and 8B) coated ETT segments (results for coatings below 1.25% w/v PLGA not shown).

### DESCRIPTION OF INVENTION

### Detailed Description of the Invention

The present invention provides a means to reduce the occurrence of infections associated with the introduction of a medical device to the body, such as ventilator-associated pneumonia (VAP), by utilizing an antimicrobial from the family of Formula (I) as part of a release coating on the medical device, such as an endotracheal tube (ETT).

In one aspect, the present invention therefore provides a composition for coating a surface, said composition comprising:
a) a polymer selected from the group: poly(lactic-co-glycolic acid), polylactide, polyglycolide, polyester, polyurethane and combinations thereof; and
b) an antimicrobial compound of Formula (I).

In one aspect of the invention, the composition for coating a surface provides a self-limiting drug release profile of the compound of Formula (I).

In a preferred form of the invention, the polymer is poly(lactic-co-glycolic acid).

In a preferred form of the invention, the polymer is biodegradable.

The composition provides a self-limiting drug release profile, via concentration dependent release of an antibiotic compound of Formula (I). The self-limiting drug release profile is preferably a self-limiting tunable drug release profile.

The control of localized antimicrobial release from medical devices has generally been through the physical mixture of the antimicrobial into a biodegradable polymer matrix. This type of drug incorporation, however, generally results in a burst of antimicrobial active agent, where large amounts of drug are released before the rate stabilizes. Burst profiles of drugs tend to be unpredictable and can lead to toxic concentrations of the active. Additionally, the maximum amount of drug loading is limited before it begins affecting the mechanical and degradation properties of the device. Furthermore, many drug-eluting polymer matrices exhibit a burst release without sustaining the drug concentration for the duration of complete polymer degradation.

In one aspect, the invention comprises a composition for coating a surface, wherein the composition comprises:
a) a polymer selected from the group: poly(lactic-co-glycolic acid), polylactide, polyglycolide, polyester, polyurethane and combinations thereof; and
b) an antimicrobial compound of Formula (I);
   wherein the concentration of the antimicrobial compound of Formula (I) in the composition is at least 0.01 mg/mL.

In a preferred form of the invention, the polymer is poly(lactic-co-glycolic acid).

In a preferred form of the invention, the polymer is biodegradable.

In a preferred form of the invention, the concentration of the antimicrobial compound of Formula (I) in the composition is at least 0.1 mg/mL. Preferably still, the concentration of the antimicrobial compound of Formula (I) in the composition is at least 1 mg/mL.

In one aspect, the invention comprises a coating, wherein the coating comprises:
a) a polymer selected from the group: poly(lactic-co-glycolic acid), polylactide, polyglycolide, polyester, polyurethane and combinations thereof; and
b) an antimicrobial compound of Formula (I);
   wherein the amount of the antimicrobial compound of Formula (I) per unit surface of the coating is at least 0.2 μg/cm².

In a preferred form of the invention, the polymer is poly(lactic-co-glycolic acid).

In a preferred form of the invention, the polymer is biodegradable.

In a preferred form of the invention, the amount of the antimicrobial compound of Formula (I) per unit surface of the coating is at least 2 μg/cm². Preferably still, the amount of the antimicrobial compound of Formula (I) per unit surface of the coating is at least 20 μg/cm².

In one aspect, the invention comprises a coating, wherein the coating comprises:
a) a polymer selected from the group: poly(lactic-co-glycolic acid), polylactide, polyglycolide, polyester, polyurethane and combinations thereof; and
b) an antimicrobial compound of Formula (I);
   and wherein, when a sample of the coating is placed into a well of tissue culture polystyrene (TOPS) 48-well plate and 500 μL of fresh 1× phosphate-buffered saline is pipetted into each well containing the sample of coating such that complete coverage of each sample of the coating with the phosphate-buffered saline is ensured, and the well of tissue culture polystyrene is sealed and placed in a shaker incubator at 37° C. (80 rpm), the concentration of the antimicrobial compound of formula (I) in the solution at 24 hours is at least 0.05 μg of the compound of Formula (I) per cm² of coating surface per mL.

In a preferred form of the invention, the polymer is poly(lactic-co-glycolic acid).

In a preferred form of the invention, the polymer is biodegradable.

In a preferred form of the invention, the concentration of antimicrobial compound in the aqueous solution after 24 hours is at least 0.5 μg/mL. Preferably still, the concentration of antimicrobial compound in the aqueous solution after 24 hours is at least 5 μg/mL.

Through in vitro experiments, the inventors have discovered that the release profile of the antimicrobial compounds from the coatings of the present invention is such that an effective or useful antimicrobial concentration of the antimicrobial compound at the surface to which the coating is applied can be attained.

In embodiments of the aforementioned coatings the concentration of the antimicrobial compound of Formula (I) at the surface to which the coating is applied is an effective inhibitory concentration for bacteria selected from the group: gram positive bacteria and gram negative bacteria and combinations thereof.

In embodiments of the aforementioned coatings the concentration of the antimicrobial compound of Formula (I) at the surface to which the coating is applied is an effective inhibitory concentration for gram positive bacteria.

In embodiments of the aforementioned coatings the concentration of the antimicrobial compound of Formula (I) at the surface to which the coating is applied is an effective inhibitory concentration for gram positive bacteria wherein the gram positive bacteria is S. aureus.

In embodiments of the aforementioned coatings the concentration of the antimicrobial compound of Formula (I) at the surface to which the coating is applied is an effective inhibitory concentration for gram negative bacteria.

In embodiments of the aforementioned coatings the concentration of the antimicrobial compound of Formula (I) at the surface to which the coating is applied is an effective inhibitory concentration for gram negative bacteria wherein the gram negative bacteria is P. aeruginosa.

In embodiments of the invention, the concentration of the antimicrobial compound at the surface to which the coating is applied is an effective inhibitory concentration for bacteria selected from the group: S. aureus and P. aeruginosa and combinations thereof.

In embodiments of the aforementioned coatings the concentration of the antimicrobial compound of Formula (I) at the surface to which the coating is applied is a useful inhibitory concentration for bacteria selected from the group: gram positive bacteria and gram negative bacteria and combinations thereof.

In embodiments of the aforementioned coatings the concentration of the antimicrobial compound of Formula (I) at the surface to which the coating is applied is a useful inhibitory concentration for gram positive bacteria.

In embodiments of the aforementioned coatings the concentration of the antimicrobial compound of Formula (I) at the surface to which the coating is applied is a useful inhibitory concentration for gram positive bacteria wherein the gram positive bacteria is S. aureus.

In embodiments of the aforementioned coatings the concentration of the antimicrobial compound of Formula (I) at the surface to which the coating is applied is a useful inhibitory concentration for gram negative bacteria.

In embodiments of the aforementioned coatings the concentration of the antimicrobial compound of Formula (I) at the surface to which the coating is applied is a useful inhibitory concentration for gram negative bacteria wherein the gram negative bacteria is P. aeruginosa.

In embodiments of the invention, the concentration of the antimicrobial compound at the surface to which the coating is applied is a useful inhibitory concentration for bacteria selected from the group: S. aureus and P. aeruginosa and combinations thereof.

Furthermore, the inventors have discovered that the release profile of the antimicrobial compounds from the coatings of the present invention is such that an effective or useful antimicrobial concentration of the antimicrobial compound at the surface to which the coating is applied can be attained for extended periods of time, perhaps even the operational lifetime of a medical device, such as an endotracheal tube.

In in vitro experiments, the inventors have demonstrated the coatings of the invention will continue to release antimicrobial compound of Formula (I) at concentrations effective to inhibit bacterial growth for extended periods of time. In particular, the inventors believe that the in vitro experiments described herein as an assessment of longevity of release are considerably more onerous than the conditions to which a coated surface of a medical device, such as an endotracheal tube, would be exposed in vivo.

Antimicrobial compounds of Formula (I) are characterised by having relatively high octanol:water or partition coefficient (LogP) values and relatively low aqueous solubility (LogS) values. For example, the antimicrobial compound of Formula E (below) has a LogP of 5.64 and a Log S of −7.33 (calculated using ALOGPS 2.1, an algorithm accessible at: http://www.vcclab.org/lab/alogps/). These LogP and LogS values suggest that the antimicrobial compounds of Formula (I) are highly lipophilic and of very low solubility in aqueous biological fluids and that they are therefore not capable of crossing membranes as a result of their high affinity to the lipid membrane. As a result, an equilibrium between the amount of antimicrobial compound released from the polymer and that present in the vicinity of the coating or device will be reached sooner translating to an even longer lasting release profile in vivo. This is in contrast to the in vitro experiments described herein where 100 μL and 500 μL of the solution was removed at various time points, effectively reducing the concentration of the drug present in the vicinity of the coating driving its release further.

In one aspect of the invention, the antimicrobial compounds of Formula (I) have LogP values of 5.4±1.5.

In one aspect of the invention, the antimicrobial compounds of Formula (I) have LogP values of 5.4±1.0.

In one aspect of the invention, the antimicrobial compounds of Formula (I) have LogP values of 5.4±0.54.

In one aspect of the invention, the antimicrobial compounds of Formula (I) have LogS values of −7.3±1.0.

In one aspect of the invention, the antimicrobial compounds of Formula (I) have LogS values of −7.3±0.5.

In one aspect of the invention, the antimicrobial compounds of Formula (I) have LogS values of −7.3±0.3.

In one aspect of the invention, the antimicrobial compounds of Formula (I) have LogS values of −7.3±0.2.

In one aspect of the invention, the antimicrobial compounds of Formula (I) have LogS values of −7.25±0.16.

In one aspect of the invention, the antimicrobial compounds of Formula (I) have LogP values of 5.4±0.5 and LogS values of −7.3±0.2.

In one aspect of the invention, the antimicrobial compounds of Formula (I) have LogP values of 5.4±0.4 and LogS values of −7.3±1.0.

In one aspect of the invention, the antimicrobial compounds of Formula (I) have LogP values of 5.4±1.0 and LogS values of −7.25±0.16.

In one form, the invention comprises a coating, wherein the coating comprises
a) a polymer selected from the group: poly(lactic-co-glycolic acid), polylactide, polyglycolide, polyester, polyurethane and combinations thereof; and
b) an antimicrobial compound of Formula (I);
   wherein, when the coating is placed in an aqueous solution, and wherein, when a sample of the coating is placed into a well of tissue culture polystyrene (TOPS) 48-well plate and 500 μL of fresh 1× phosphate-buffered saline is pipetted into each well containing the sample of coating such that complete coverage of each sample of the coating with the phosphate-buffered saline is ensured, and the well of tissue culture polystyrene is sealed and placed in a shaker incubator at 37° C. (80 rpm), and wherein at 1, 4, 24 and 48 h, 100 μL of solution was removed, and wherein at 72 h and at 8 and 24 days all 500 μL of solution was removed and replaced with 500 μL of fresh 1× phosphate-buffered saline, the concentration of antimicrobial compound in the aqueous solution after 31 days is at least 5 μg of the compound of Formula (I) per cm² of coating surface per mL.

In a preferred form of the invention, the polymer is poly(lactic-co-glycolic acid).

In a preferred form of the invention, the polymer is biodegradable.

In a preferred form of the invention, the concentration of antimicrobial compound in the aqueous solution after 31 days is at least 8 μg of the compound of Formula (I) per cm² of coating surface per mL. Preferably still, the concentration of antimicrobial compound in the aqueous solution after 31 days is at least 10 μg of the compound of Formula (I) per cm² of coating surface per mL.

The period for which coatings of the invention are capable of providing an effective or useful inhibitory concentration of the compound of Formula (I) is a function of both the concentration of the antimicrobial compound of Formula (I) and the thickness of the coating comprising the antimicrobial compound and the polymer, such as PLGA. The inventors have discovered that it is possible to produce a coating of the invention using the compounds of Formula (I) and the polymer, such as PLGA that provides an effective or useful inhibitory concentration of the compound of Formula (I) for extended periods.

In a preferred form of the invention, the thickness of the coating is at least 4 μm and the concentration of the compound of Formula I is at least 25 μg/cm².

The inventors have further discovered that the compositions of the present invention are advantageous to produce the coatings of the present invention.

These discoveries are a function of a number of heretofore unknown properties arising from the interaction of the antimicrobial compounds of Formula (I), such as the compound of Formula E, with polymers such as PLGA, including the low solubility and high lipophilicity of the antimicrobial compounds of Formula (I), the stability of mixtures of antimicrobial compounds of Formula (I) and polymers such as PLGA, the rate of release of antimicrobial compounds of Formula (I) from polymers such as PLGA, and the inhibitory concentration of the antimicrobial compounds of Formula (I) at the surface of coatings of the invention.

Without wishing to be bound by theory, the inventors consider that hydrogen bonding and partitioning interactions between polymers such as PLGA and the compounds of Formula (I), such as the compound of Formula E, play an important role in the release of compounds of Formula (I) from the polymer. On one hand there is the release of the compounds of Formula (I) from a high concentration in the polymer to a low concentration in the environment due to diffusion. On the other hand an opposing force is the H-bonding between polymers such as PLGA and the compounds of Formula (I) pulling back on the compound of Formula (I) and thereby resisting its release due to diffusion. A further interaction contributing to the controlled release of antimicrobial compounds of Formula (I) from polymers such as PLGA is the tendency of such polymers to form internal hydrophobic pockets favourable to the retention of compounds of Formula (I) due to the generally high lipophilicity of compounds of Formula (I). On this basis, it is to be understood that similarly advantageous results will be observed from coatings comprising compounds of Formula (I) with other polymers having a similar hydrogen-bonding and hydrophobic pocket forming capacity, such as polylactide, polyglycolide, polyester, polyurethane and combinations thereof, and combinations of PLGA with polylactide, polyglycolide, polyester, polyurethane.

In a preferred form of the invention, the coating is applied to a surface wherein the surface is substantially polyvinyl chloride. In a preferred form of the invention, the surface is polyvinyl chloride.

In a preferred form of the invention, the surface forms part of a medical device.

In a preferred form of the invention, the medical device is an endotracheal tube.

An antimicrobial compound of Formula (I) is a compound having a structure selected from Group I, wherein Group I consists of:

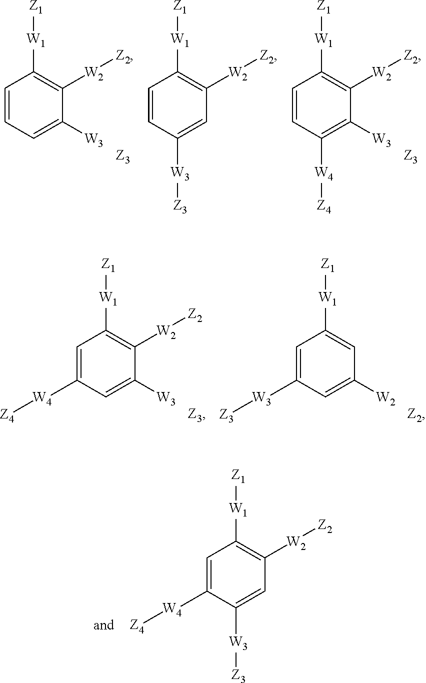

wherein
each of W₁, W₂, W₃, and W₄ is the same and is selected from the group consisting of C₂₋₄ alkyl, substituted C₂₋₄ alkyl; and C₂ alkene;
each of Z₁, Z₂, Z₃, and Z₄ is the same and each is selected from the group consisting of:

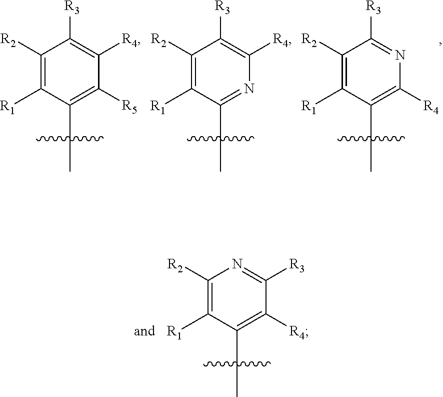

each of R₁, R₂, R₃, R₄, and R₅ is independently H or C₁₋₈ heteroalkyl, wherein the C₁₋₈ heteroalkyl comprises —CO₂H or an ester thereof, with the proviso that at least one of R₁, R₂, R₃, R₄, and R₅ is C₁₋₈ heteroalkyl, or a pharmaceutically acceptable salt thereof.

In one aspect of the present invention, the compounds of Formula (I) are compounds in which the C₁ heteroalkyl group of Formula (I) is —CO₂H or an ester thereof.

Antimicrobial compounds of Formula (I) belong to a new class of styrylbenzene-based derivative antibiotics. This class of antimicrobial compounds has shown activity against the Mechanosensitive Ion Channel of Large Conductance (MscL), a novel and highly sought after bacterial target.

MscL is a highly conserved transmembrane protein found in all bacteria but not in the human genome, making it an ideal drug target. The channel is responsible for saving bacterial cells from lysis in a high osmotic environment. It responds to a high turgor pressure by opening up and allowing bacteria to release osmolytes thereby reducing the pressure within.

Styrylbenzene-based antibiotics lower the threshold at which these channels open and elongate their opening times, causing the loss of important osmolytes and other biomolecules and thereby weakening the bacteria. For the purpose of utilizing a compound in an antimicrobial coating, properties such as high chemical and thermal stability and the ability of large scale and cost-effective manufacturing, are critical for the success of such an application.

The antimicrobial compounds of Formula (I) have these properties, making them an attractive antimicrobial compound for incorporation in polymer coatings.

The antimicrobial compounds of Formula (I) have demonstrated their effectiveness as antimicrobially active molecules, targeting Mscl in both gram positive and gram negative bacteria.

Preferably the antimicrobial compound of Formula (I) is chosen from the following:

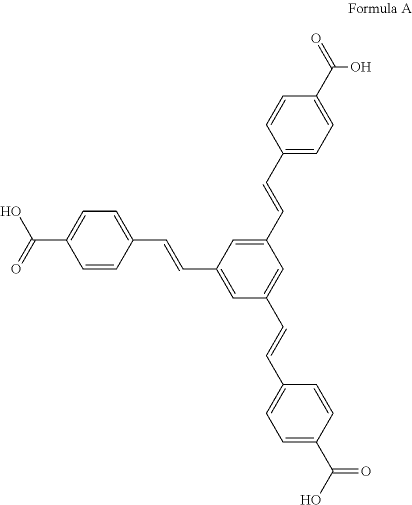 [SMILES: O=C(O)c1ccc(/C=C/c2cc(/C=C/c3ccc(C(=O)O)cc3)cc(/C=C/c3ccc(C(=O)O)cc3)c2)cc1]

or pharmaceutically acceptable salts thereof; and/or Formula B:

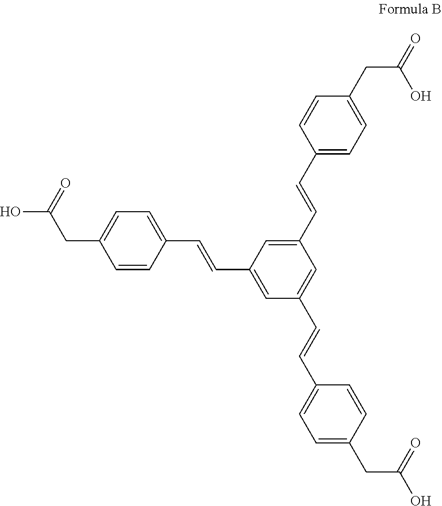 [SMILES: O=C(O)Cc1ccc(/C=C/c2cc(/C=C/c3ccc(CC(=O)O)cc3)cc(/C=C/c3ccc(CC(=O)O)cc3)c2)cc1]

or pharmaceutically acceptable salts thereof; and/or Formula C:

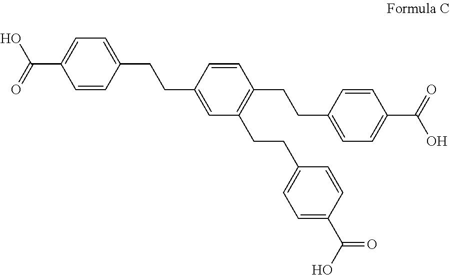 [SMILES: O=C(O)c1ccc(CCc2ccc(CCc3ccc(C(=O)O)cc3)c(CCc3ccc(C(=O)O)cc3)c2)cc1]

or pharmaceutically acceptable salts thereof; and/or Formula D:

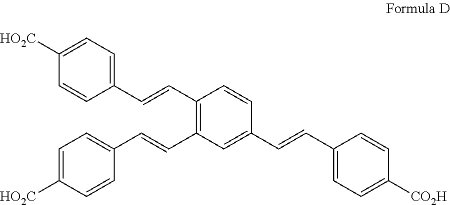 [SMILES: O=C(O)c1ccc(/C=C/c2ccc(/C=C/c3ccc(C(=O)O)cc3)c(/C=C/c3ccc(C(=O)O)cc3)c2)cc1]

or pharmaceutically acceptable salts thereof; and/or Formula E:

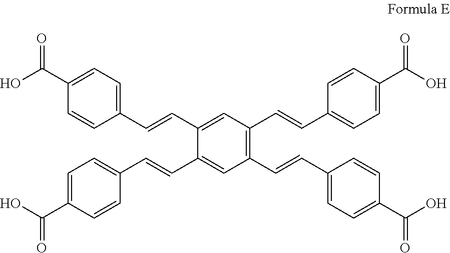 [SMILES: O=C(O)c1ccc(/C=C/c2cc(/C=C/c3ccc(C(=O)O)cc3)c(/C=C/c3ccc(C(=O)O)cc3)cc2/C=C/c2ccc(C(=O)O)cc2)cc1]

or pharmaceutically acceptable salts thereof.

Most preferably, the antimicrobial compound is Formula E (also referred herein to as BCP3).

The synthesis, antimicrobial properties and cytotoxicity of compounds of Formula (I) have been previously examined and published in US 2015/0189873 and in Chem. Eur. J. 2013, 19, 17980-17988, the disclosures of both of which are incorporated herein in their entirety.

The terms “drug”, “active agent”, “antibiotic” and “antimicrobial” are used interchangeably in the present specification. The terms generally refer to the antibiotic compounds of Formula (I). However, the term may also refer to other active agents (such as additional antibiotics etc.) that may be incorporated into the coatings of the present invention.

Poly(lactic-co-glycolic acid) (PLGA) is a co-polymer of glycolic and lactic acids, with each of the monomeric units being linked by ester linkages providing biodegradable properties. PLGA has been approved by the FDA.

Without wishing to be bound by theory, the use of polymers such as PLGA in embodiments of the present invention serves two purposes; not only does it act to hold the antibiotic in place on the medical device, it also allows the antibiotic to be released both by diffusion and, in the longer term, via the degradation of the polymer chains. Furthermore, and still without wishing to be bound by theory, the hydrophobic nature of polymers such as PLGA allows for a slower sustained release of antibiotic in aqueous environments.

PLGA is synthesized by means of ring-opening co-polymerization of two different monomers, the cyclic dimers (1,4-dioxane-2,5-diones) of glycolic acid and lactic acid. Polymers can be synthesized as either random or block copolymers thereby imparting additional polymer properties. Depending on the ratio of lactide to glycolide used for the polymerization, different forms of PLGA can be obtained: these are usually identified in regard to the molar ratio of the monomers used (e.g. PLGA 75:25 identifies a copolymer whose composition is 75% lactic acid and 25% glycolic acid).

PLGA degrades by hydrolysis of its ester linkages in the presence of water. It has been shown that the time required for degradation of PLGA is related to the monomers' ratio used in production: the higher the content of glycolide units, the lower the time required for degradation as compared to predominantly lactide materials. An exception to this rule is the copolymer with 50:50 monomers ratio which exhibits the faster degradation (about two months). In addition, polymers that are end-capped with esters (as opposed to the free carboxylic acid) demonstrate longer degradation half-lives. Preferably, where the polymer is PLGA, the PLGA of the composition and coating of the present invention is a blend of end-capped and uncapped PLGA, wherein said end-capped polymer has terminal residues functionalized as esters and said uncapped polymer has terminal residues existing as carboxylic acids. Preferably the PLGA is biocompatible and/or biodegradable. Preferably the PLGA has a molar ratio of the monomers used of 75% poly(D,L-lactide) and 25% glycolide.

Other biodegradable polymers such as polylactide, polyglycolide, polyester, and polyurethane exhibit similar behaviours and properties to PLGA in that they all contain hydrolysable and/or enzyme cleavable groups along the main chain of the polymer, allowing them to be broken down into non-toxic oligomers and monomers able to be excreted naturally, and also in that these polymers all tend to form internal hydrophobic pockets, capable of retaining lipophilic compounds of Formula (I). In this way, such biodegradable polymers enable controlled release of antimicrobial compounds of Formula (I) from the coatings of the present invention.

The use of biodegradable polymers such as polylactide, polyglycolide, polyester, and polyurethane, as well as their corresponding copolymers such as PLGA for the controlled release of drug compounds has been studied and reported on in the scientific literature in journal articles including Nair, Lakshmi S. and Laurencin, Cato T. “Biodegradable polymers as biomaterials”, Progress in Polymer Science, 2007, Vol. 32(8), pp. 762-798, and Kamaly, Nazila; Yameen, Basit; Wu, Jun and Farokhzad, Omid C. “Degradable Controlled-Release Polymers and Polymeric Nanoparticles: Mechanisms of Controlling Drug Release”, Chemical reviews, 24 Feb. 2016, Vol. 116(4), pp. 2602-63, the disclosures of which are hereby incorporated in their entirety.

Furthermore, in some embodiments, the composition of the present invention may produce unexpected results in the form of a self-limiting drug release profile, i.e. as the levels of released antimicrobial increase, they reach a plateau; then as the concentration of the antimicrobial in the immediate environment is reduced (due to diffusion, metabolism etc.), release of the antimicrobial is resumed.

Previously, PLGA has only shown constant release of actives when used as a coating on medical devices. The release profile of actives combined with PLGA has previously been reported to be based on the degradation rate of the PLGA, with some contribution from water penetration and solubilisation of the PLGA (Hines et al Crit Rev Ther Drug Carrier Syst. 2013 30(3): 257-276).

In contrast, certain embodiments of the present invention may provide a self-limiting drug release profile, via concentration dependent release that develops uniquely from the combination of the polymer, preferably PLGA, and the antibiotics chosen.

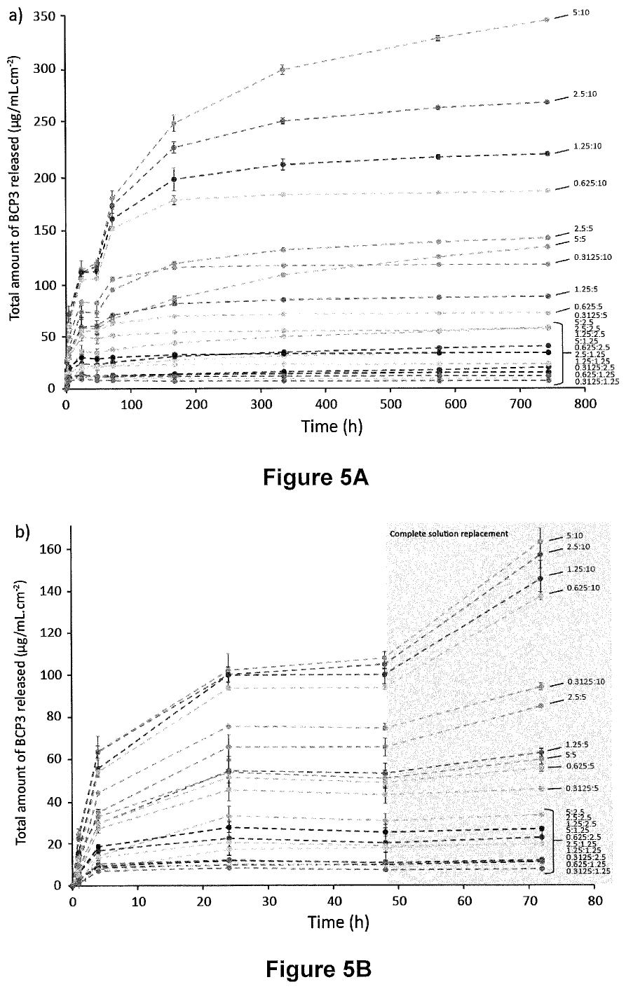

A self-limiting drug release profile, via concentration dependent release is defined as an initial increase in antimicrobial active agent release from the polymer, matrix, followed by the reaching of a release plateau where no further antimicrobial active agent is released. Then, as the concentration of the antimicrobial in the immediate environment is reduced (due to diffusion, metabolism etc.), release of the antimicrobial is resumed. An example of the release profile generated during a self-limiting concentration dependent release is shown in FIG. 5B. Here, the initial release of active causes an increase in drug concentration for some hours, before the drug concentration plateaus. Replacement of the test medium, equivalent to degradation of the antibiotic in the medium surrounding an inserted medical device, causes a second release of active agent. Subsequent replacement/degradation cycles can be carried out.

The self-limiting drug release profile can be “tuned” to make a self-limiting tunable drug release profile by manipulating the thickness of the coating, the concentration of the antibiotic and/or the ratio of the monomers used in the polymer.

Preferably, the polymer is applied to the medical device at a concentration of between 0.1% w/v and 10% w/v; for example between 0.2% w/v and 10% w/v, 0.5% w/v and 9% w/v, 1.0% w/v and 7% w/v, 0.3125% w/v and 5% w/v, 0.1% w/v and 2.5% w/v; preferably 5.0, 2.5, 1.25, 0.625 or 0.3125% w/v polymer. Preferably, the polymer is PLGA.

Preferably, the antimicrobial compound of Formula (I) is applied to the medical device at a concentration of between 1 mg/mL and 15 mg/mL; for example between 1.25 mg/mL and 10 mg/mL, 1.25 mg/mL and 5 mg/mL, 2.5 mg/mL and 5 mg/mL, 2.5 mg/mL and 10 mg/mL; preferably 10, 5, 2.5 or 1.25 mg/mL antimicrobial compound of Formula (I).

Preferably, the amounts of polymer, and Formula (I) in the composition of the invention to be applied to a surface of a medical device to form a coating of the invention are chosen such that the antimicrobial compound is able to be released from the medical device for the term of use of the device (e.g. the ETT). The self-limiting drug release profile may involve multiple cycles of antibiotic release, followed by plateaus where the antibiotic is not released until the concentration drops in the surrounding environment, at which point the release of antibiotic from the coating recommences.

For example, the self-limiting drug release profile may provide an initial release time of between 12 h and 48 h, followed by a plateau of between of between 12 h and 48 h, followed by a second release time of between 12 h and 48 h, followed by a second plateau of between of between 12 h and 48 h, etc. Preferably, the self-limiting drug release profile provides an initial release time of about 24 h, followed by a plateau of about 24 h, followed by a second release time of about 24 h, followed by a second plateau of about 24 h, etc. this release and plateau may continue for between 1 and 30 days.

Preferably, the compound of Formula (I) is present in an amount effective to reduce or inhibit bacterial infection associated with the medical device. Preferably, the coating releases the compound of Formula (I) in an amount effective to reduce or inhibit the likelihood of infection associated with the insertion of the medical device in a subject.

Preferably, the coating releases the compound of Formula (I) in concentrations effective to reduce or inhibit infection associated with the medical device for a period ranging from 1 to 30 days.

The polymer, allows the antimicrobial compound of Formula (I) to be bound in the coating. With higher polymer concentrations, more antimicrobial can be incorporated and held together by the polymer following the coating process. There is preferably a linear relationship between the concentration of polymer and the concentration of antimicrobial in the coating solution. By varying the antimicrobial and polymer ratios and concentrations, the antimicrobial loading in the coating can be varied as desired, as can the release profile.

The polymer/Formula (I) composition may be applied as one coat, two coats or more coats. The ratio of polymer to compound of Formula (I) may be different in different layers to change or tune the self-limiting release profile of the antibiotic.

Preferably, the coating is applied at a thickness of between 1 μm to 50 μm, for example between 2 μm to 40 μm, 4 μm to 20 μm; preferably 2 μm, 4 μm, 6 μm, 8 μm, 10 μm, 12 μm, 14 μm, 16 μm, 18 μm, or 20 μm.

The polymer, (containing the compound of Formula (I)) may be coated onto the medical device over the entire surface of the medical device, or over at least 20%, 30%, 40%, 50% 60% 70% 80% or 90%, 95% or 99% of the medical device.

The polymer, (containing the compound of Formula (I)) may be coated onto the medical device by any suitable method, such as dipping or spraying. The polymer, may be applied as a paste or foam, optionally by painting the polymer, onto the medical device.

As medical devices such as stents, catheters, endotracheal tube and tracheostomy tubes are made in a variety of configurations and sizes, the exact dose administered will vary with device size, surface area and design. However, certain principles can be applied in the application of this art. Drug dose can be calculated as a function of dose per unit area (of the portion of the device being coated), total drug dose administered can be measured and appropriate surface concentrations of active drug can be determined.

The release profile of the antimicrobial compound of Formula (I) is preferably initially via diffusion, followed by a combination of diffusion and release via the polymer, degradation owing to the degradable nature of the polymer, as a result of the ester linkages present in the polymer backbone.

The polymer, and the compound of Formula (I) are preferably provided in a solution of tetrahydrofuran (THF), so that they may then then be used to coat the medical device. Other suitable solvents include chloroform, dichloromethane, acetone, etc.

Other active agents may also be incorporated into the composition of the present invention. For example, additional antimicrobial agents such as antibacterials, antifungals etc.; lubricating agents; agents that reduce biofouling; may be incorporated in the coating.

Additional antimicrobial agents that can be used include, but are not limited to silver compounds (e.g., silver chloride, silver nitrate, silver oxide), silver ions, silver particles, iodine, povidone/iodine, chlorhexidine, 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin, vancomycin, azaserine, candicidin(s), chlorphenesin, dermostatin(s), filipin, fungichromin, mepartricin, nystatin, oligomycin(s), ciproflaxacin, norfloxacin, ofloxacin, pefloxacin, enoxacin, rosoxacin, amifloxacin, fleroxacin, temafloaxcin, lomefloxacin, perimycin A or tubercidin, and the like.

Whilst the Examples generally refer to the device to be coated as an endotracheal tube (ETT), there is no desire to limit this invention to only these devices. Other implantable or insertable medical devices (which may be removable) are also encompassed by the presently claimed coating method and composition. The terms “implantable” and “insertable” are used interchangeably. The medical devices may be bioabsorbable and/or removable. Examples of implantable or insertable medical devices include tracheostomy tubes, catheters, guide wires, balloons, filters, stents (including sinus stents, urethral and ureteral stents), stent grafts, vascular grafts, vascular patches, tympanostomy tubes, prosthetic sphincters (including bladder sphincters), and shunts. Among medical devices in accordance with the present invention are biliary, ureteral and pancreatic stents, stent covers, catheters, venous access devices and devices bridging or providing drainage between a sterile and non-sterile body environment or between two sterile body environments. The implantable or insertable medical device may be adapted for implantation or insertion into, for example, the coronary vasculature, peripheral vascular system, oesophagus, trachea, colon, biliary tract, urinary tract, prostate or brain.

Preferably, the medical device is constructed, extruded or formed before coating with the polymer, and antimicrobial compound of Formula (I).

The insertable medical devices can be formed from various materials, such as polymeric and/or metallic materials, and may be non-degradable or biodegradable. The material, polymeric and/or metallic, that makes up the medical device before coating with the polymer and antibiotic will be referred to the as the “medical device material”.

Preferred substantially non-biodegradable biocompatible medical device materials include thermoplastic and elastomeric polymeric materials. Polyolefins such as metallocene catalyzed polyethylenes, polypropylenes, and polybutylenes and copolymers thereof; vinyl aromatic polymers such as polystyrene; vinyl aromatic copolymers such as styrene-isobutylene copolymers and butadiene-styrene copolymers; ethylenic copolymers such as ethylene vinyl acetate (EVA), ethylene-methacrylic acid and ethylene-acrylic acid copolymers where some of the acid groups have been neutralized with either zinc or sodium ions (commonly known as ionomers); polyacetals; chloropolymers such as polyvinylchloride (PVC); fluoropolymers such as polytetrafluoroethylene (PTFE); polyesters such as polyethyleneterephthalate (PET); polyester-ethers; polyamides such as nylon 6 and nylon 6,6; polyamide ethers; polyethers; elastomers such as elastomeric polyurethanes and polyurethane copolymers; silicones; polycarbonates; and mixtures and block or random copolymers of any of the foregoing are non-limiting examples of non-biodegradable biocompatible medical device materials useful for manufacturing the medical devices of the present invention.

Among particularly preferred non-biodegradable medical device materials are polyolefins, ethylenic copolymers including ethylene vinyl acetate copolymers (EVA) and copolymers of ethylene with acrylic acid or methacrylic acid; elastomeric polyurethanes and polyurethane copolymers; metallocene catalyzed polyethylene (mPE), mPE copolymers, ionomers, and mixtures and copolymers thereof; and vinyl aromatic polymers and copolymers. Among preferred vinyl aromatic copolymers are included copolymers of polyisobutylene with polystyrene or polymethylstyrene, even more preferably polystyrene-polyisobutylene-polystyrene triblock copolymers. These polymers are described, for example, in U.S. Pat. Nos. 5,741,331, 4,946,899 and U.S. Ser. No. 09/734,639, each of which is hereby incorporated by reference in its entirety. Ethylene vinyl acetate having a vinyl acetate content of from about 19% to about 28% is an especially preferred non-biodegradable material. EVA copolymers having a lower vinyl acetate content of from about 3% to about 15% are also useful in particular embodiments of the present invention as are EVA copolymers having a vinyl acetate content as high as about 40%. These relatively higher vinyl acetate content copolymers may be beneficial in offsetting stiffness from coating layers. Among preferred elastomeric polyurethanes are block and random copolymers that are polyether based, polyester based, polycarbonate based, aliphatic based, aromatic based and mixtures thereof. Commercially available polyurethane copolymers include, but are not limited to, Carbothane®, Tecoflex®, Tecothane®, Tecophilic®, Tecoplast®, Pellethane®, Chronothane® and Chronoflex®. Other preferred elastomers include polyester-ethers, polyamide-ethers and silicone.

Preferred biodegradable medical device materials include, but not limited to, polylactic acid, polyglycolic acid and copolymers and mixtures thereof such as poly(L-lactide) (PLLA), poly(D,L-lactide) (PLA); polyglycolic acid [polyglycolide (PGA)], poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), poly(D,L-lactide-co-caprolactone) (PLA/PCL), poly(glycolide-co-caprolactone) (PGA/PCL); polyethylene oxide (PEO), polydioxanone (PDS), polypropylene fumarate, poly(ethyl glutamate-co-glutamic acid), poly(tert-butyloxy-carbonylmethyl glutamate), polycaprolactone (PCL), polycaprolactone co-butylacrylate, polyhydroxybutyrate (PHBT) and copolymers of polyhydroxybutyrate, poly(phosphazene), polyphosphate ester), poly(amino acid) and poly(hydroxy butyrate), polydepsipeptides, maleic anhydride copolymers, polyphosphazenes, polyiminocarbonates, poly[(97.5% dimethyl-trimethylene carbonate)-co-(2.5% trimethylene carbonate)], cyanoacrylate, polyethylene oxide, hydroxypropylmethylcellulose, polysaccharides such as hyaluronic acid, chitosan and regenerate cellulose, and proteins such as gelatine and collagen, and mixtures and copolymers thereof, among others.

As used herein, the term “heteroalkyl” is understood to include —CO₂H or an ester thereof as a C₁ heteroalkyl.

As used herein, a “therapeutic agent” refers to any substance that, when administered in a therapeutically effective amount to a patient suffering from a disease, has a therapeutic beneficial effect on the health and well-being of the patient. A therapeutic beneficial effect on the health and well-being of a patient includes, but it not limited to: (1) curing the disease; (2) slowing the progress of the disease; (3) causing the disease to retrogress; or, (4) alleviating one or more symptoms of the disease. As used herein, a “therapeutic agent” also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a retrogressed level once such level has been achieved by a therapeutically effective amount of a substance, which may be the same as or different from the substance used in a prophylactically effective amount; or, (3) preventing or delaying recurrence of the disease after a course of treatment with a therapeutically effective amount of a substance, which may be the same as or different from the substance used in a prophylactically effective amount, has concluded. As used herein, the term “therapeutic agent” is used interchangeably with the term “drug.”

As used herein, “treating” refers to the administration of a therapeutically effective amount of a therapeutic agent to a patient known or suspected to be suffering from a disease, such as a microbial infection.

As used herein, a “therapeutically effective amount” refers to that amount of a therapeutic agent that will have a beneficial effect, which may be curative or palliative, on the health and well-being of the patient with regard to the disease (e.g., infection) with which the patient is known or suspected to be afflicted. A therapeutically effective amount may be administered as a single bolus, as intermittent bolus charges, or as short, medium or long term sustained release formulations or as any combination of these. As used herein, short-term sustained release refers to the administration of a therapeutically effective amount of a therapeutic agent over a period from about several hours to about 3 days. Medium-term sustained release refers to administration of a therapeutically effective amount of a therapeutic agent over a period from about 3 days to about 14 days and long-term refers to the delivery of a therapeutically effective amount over any period in excess of about 14 days.

As used herein, a “subject” refers to any species that might benefit from treatment using the method herein but at present preferably a mammal and most preferably a human being.

As used herein, and unless otherwise specified, the terms “polymer” and “polymeric” refer to compounds that are the product of a polymerization reaction. These terms are inclusive of homopolymers (i.e., polymers obtained by polymerizing one type of monomer), copolymers (i.e., polymers obtained by polymerizing two or more different types of monomers), terpolymers, etc., including random, alternating, block, graft, dendritic, crosslinked, and any other variations of polymers. The terms are inclusive of a polymer blend of two or more polymers, for example, three, four, five, six, seven, eight, nine, and ten polymers. The polymers in the blend can be of various ratios. For example, in a two polymer blend, the amount of one polymer can vary from 0.5% to 99.5% by weight, and the other polymer can vary from 99.5% to 0.5% by weight.

As used herein, the terms “bioabsorbable,” “bioresorbable” “bioerodable,” and “biodegradable” can be used interchangeably. By “bioabsorbable” or “bioresorbable,” when used with reference to a polymer, it is meant that a polymer, a polymeric scaffold, a polymeric substrate, or a polymeric coating can, for example, be absorbed by a subject's body. By “biodegradable” when used with reference to a polymer, it is meant that a polymer, a polymeric scaffold, a polymeric substrate, or a polymeric coating is susceptible to degradation or lowering of molecular weight by a living system and can be disposed of in a subject's body. Biodegradation as used herein may occur through hydrolysis, enzymatic reactions, oxidation, and other chemical reactions. Bioabsorption or biodegradation can take place over a relatively short period of time, for example, 1-6 months, or an extended period of time, for example over 6 months, under physiological conditions.

As used herein, a “biostable” polymer or coating refers to a polymer or coating that is not biodegradable, which is defined above. The term “biostable” is used interchangeably with the term “non-degradable”.

The invention further provides an insertable medical device, wherein the insertable medical device comprises a coating comprising as described herein.

Preferably, the coating releases the compound of Formula (I) in an amount effective to reduce or inhibit the likelihood of infection associated with the insertion of the medical device in a subject.

The invention further provides an insertable medical device, wherein the insertable medical device comprises a coating comprising (i) a polymer selected from the group: poly(lactic-co-glycolic acid), polylactide, polyglycolide, polyester, polyurethane and combinations thereof; and (ii) 0.001 μg per mm² to 0.5 mg per mm² of the antimicrobial compound of Formula (I) of the surface area of the portion of the medical device to which the of the antimicrobial compound of Formula (I) is applied, wherein the coating on the medical device wherein the composition provides a self-limiting drug release profile of the compound of Formula (I) and wherein the coating releases the compound of Formula (I) in an amount effective to reduce or inhibit the likelihood of infection associated with the insertion of the medical device in a subject.

In a preferred form of the invention, the polymer is a biodegradable polymer.

In a preferred form of the invention, the polymer is poly(lactic-co-glycolic acid).

Preferably, the polymer, is applied to the medical device at a concentration of between 0.1% w/v and 10% w/v; for example between 0.2% w/v and 10% w/v, 0.5% w/v and 9% w/v, 1.0% w/v and 7% w/v, 0.3125% w/v and 5% w/v, 0.1% w/v and 2.5% w/v; preferably 5.0, 2.5, 1.25, 0.625 or 0.3125% w/v PLGA.

Preferably, the antimicrobial compound of Formula (I) is applied to the medical device at a concentration of between 1 mg/mL and 15 mg/mL; for example between 1.25 mg/mL and 10 mg/mL, 1.25 mg/mL and 5 mg/mL, 2.5 mg/mL and 5 mg/mL, 2.5 mg/mL and 10 mg/mL; preferably 10, 5, 2.5 or 1.25 mg/mL antimicrobial compound of Formula (I).

The dose per unit area (i.e. the amount of drug as a function of the surface area of the portion of the medical device to which drug is applied and/or incorporated) should fall within the range of 0.001 μg per mm² to 0.5 mg per mm², preferably 0.01 μg per mm² to 50 μg per mm², more preferably 0.1 μg per mm² to 5 μg per mm² of surface area. In a particularly preferred embodiment, the antimicrobial compound of Formula (I) should be applied to the surface of the medical device at a dose of 0.1 μg/mm²-10 μg/mm².

Preferably, the amounts of polymer and Formula (I) applied to the medical device are chosen such that the antimicrobial is able to be released from the medical device for the term of use of the device (e.g. the ETT). This may involve multiple cycles of antibiotic release, followed by plateaus where the antibiotic is not released until the concentration drops in the surrounding environment, at which point the release of antibiotic from the polymer coating recommences.

For example, the initial release time may be for between 12 h and 48 h, followed by a plateau of between of between 12 h and 48 h, followed by a second release time of between 12 h and 48 h, followed by a second plateau of between of between 12 h and 48 h, etc. Preferably, the initial release time is about 24 h, followed by a plateau of about 24 h, followed by a second release time of about 24 h, followed by a second plateau of about 24 h, etc.

Preferably, the compound of Formula (I) is present in an amount effective to reduce or inhibit bacterial infection associated with the medical device. Preferably, the coating releases the compound of Formula (I) in an amount effective to reduce or inhibit the likelihood of infection associated with the insertion of the medical device in a subject.

Preferably, the polymer releases the compound of Formula (I) in concentrations effective to reduce or inhibit infection associated with the medical device for a period ranging from 1 to 30 days.

The Polymer/Formula (I) composition may be applied as one coat, two coats or more coats.

Preferably, the coating is applied at a thickness of between 1 μm to 50 μm, for example between 2 μm to 40 μm, 4 μm to 20 μm; preferably 2 μm, 4 μm, 6 μm, 8 μm, 10 μm, 12 μm, 14 μm, 16 μm, 18 μm, or 20 μm.

The polymer, (containing the compound of Formula (I)) may be coated onto the medical device over the entire surface of the medical device, or over at least 20%, 30%, 40%, 50% 60% 70% 80% or 90%, 95% or 99% of the medical device.

The polymer, (containing the compound of Formula (I)) may be coated onto the medical device by any suitable method, such as dipping or spraying. The polymer, may be applied as a paste or foam, optionally by painting the polymer, onto the medical device.

The invention further provides a method of manufacturing an insertable medical device, comprising the steps of:
i) coating a surface of the device with a composition as described herein.

The invention further provides a method of manufacturing an insertable medical device, comprising the steps of:
i) providing a composition comprising:
   a) a polymer selected from the group: poly(lactic-co-glycolic acid), polylactide, polyglycolide, polyester, polyurethane and combinations thereof; and
   b) a compound of Formula (I);
ii) coating the device with the composition, such that the compound of Formula (I) is at 0.001 μg per mm² to 0.5 mg per mm².

In one form of the invention the composition provides a self-limiting drug release profile of the compound of Formula (I) wherein the coating releases the compound of Formula (I) in an amount effective to reduce or inhibit the likelihood of infection associated with the insertion of the medical device in a subject.

In a preferred form of the invention, the polymer is a biodegradable polymer.

In a preferred form of the invention, the polymer is poly(lactic-co-glycolic acid).

The polymer, (containing the compound of Formula (I)) may be coated onto the medical device over the entire surface of the medical device, or over at least 20%, 30%, 40%, 50% 60% 70% 80% or 90% of the medical device.

The polymer, (containing the compound of Formula (I)) may be coated onto the medical device by any suitable method, such as dipping, painting or spraying.

The polymer, (containing the compound of Formula (I)) may be administered in one layer, or several layers. The ratio of polymer to compound of Formula (I) may be different in different layers to change or tune the release profile of the antibiotic.

The present invention further provides a method of treating or preventing bacterial infections on insertable medical devices inserted into a subject, comprising the step of:
i) inserting a medical device coated with a composition comprising:
   a) a polymer selected from the group: poly(lactic-co-glycolic acid), polylactide, polyglycolide, polyester, polyurethane and combinations thereof; and
   b) a compound of Formula (I)
      into the subject.

In one aspect the medical device coated with the composition of the invention provides a self-limiting drug release profile of the compound of Formula (I).

In a preferred form of the invention, the polymer is poly(lactic-co-glycolic acid) (PLGA).

In a preferred form of the invention, the polymer is biodegradable.

The present invention further provides a method of treating or preventing bacterial infections on insertable medical devices inserted into a subject, comprising the step of:
i) inserting a medical device coated with a composition comprising:
   a) a polymer selected from the group: poly(lactic-co-glycolic acid), polylactide, polyglycolide, polyester, polyurethane and combinations thereof; and
   b) a compound of Formula (I) at a concentration of 0.001 uμg per mm² to 0.5 mg per mm² of surface area
      into the subject.

In one form of the invention the medical device coated with the composition of the invention provides a self-limiting drug release profile of the compound of Formula (I) wherein the coating releases the compound of Formula (I) in an amount effective to reduce or inhibit the likelihood of infection associated with the insertion of the medical device in the subject.

In a preferred form of the invention, the polymer is poly(lactic-co-glycolic acid) (PLGA).

In a preferred form of the invention, the polymer is biodegradable.

### General

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

As used herein the term “derived” and “derived from” shall be taken to indicate that a specific integer may be obtained from a particular source albeit not necessarily directly from that source.

As used herein, the singular forms “a,” “an” and “the” include plural references unless the context clearly dictates otherwise.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other than in the operating example, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the specification and claims are approximations that may vary depending upon the desired properties sought to be obtained by the present invention. Hence “80%” means “about 80%” and also “80%”. At the very least, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value; however, inherently contains certain errors necessarily resulting from the standard deviation found in their respective testing measurements

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Statistical analysis: A minimum of three experimental repeats (n 3, unless otherwise specified) were used in each study and the results are presented as mean±standard error. Statistical significance was determined by an independent Student's t-test, and a confidence level of 95% (p<0.05) was considered to be statistically significant unless otherwise stated.

### Example 1

### Preparation of BCP3-PLGA Coatings on ETT Segments

For medical devices that are widely used, such as endotracheal tubes, an antimicrobial coating would need to be easily applicable using processes that can be adapted for large scale manufacturing. Dip-coating is a robust and simple system that has been explored for the preparation of coatings for a variety of medical applications, as reported in H. Gollwitzer, K. Ibrahim, H. Meyer, W. Mittelmeier, R. Busch, A. Stemberger, “Antibacterial poly(D,L-lactic acid) coating of medical implants using a biodegradable drug delivery technology”, Journal of Antimicrobial Chemotherapy 2003, 51, pp 585-591, and in M. Zilberman, J. J. Elsner, “Antibiotic-eluting medical devices for various applications”, Journal of Controlled Release, 2008, 130, pp 202-215, the disclosures of which are hereby incorporated in their entirety.

In the case of ETTs, coating both the internal and outside surface of the ETT would maximize the defence against bacterial infection, hence a dip coating process was employed due to its simplicity and accessibility.

BCP3-PLGA Coating of ETT Segments:

Medline 7.5 mm internal diameter poly(vinyl chloride) (PVC) endotracheal tubes (ETT) (Medline Industries, IL, USA) were cut into 5 mm ring segments using a fresh scalpel to be used in the coating process. BCP3 (Boulos & Cooper Pharmaceuticals, WA, Australia) and poly(D,L-lactide-co-glycolide) (PLGA)(Sigma-Aldrich, lactide:glycolide 75:25, Mw 76,000-115,000 Da) was dissolved/suspended in tetrahydrofuran (THF). The solutions were then sonicated for 15 mins using a bath sonicator. The final solutions obtained were combinations of 10, 5, 2.5 and 1.25 mg/mL BCP3 and 5, 2.5, 1.25, 0.625, 0.3125% w/v of PLGA. Previously prepared ETT segments were then dipped into 3 mL solutions of PLGA:BCP3 combinations for 10 s and removed using fine forceps and allowed to dry in a laminar flow cabinet for 5 min prior to being dipped again in their respective solutions for another 10 s.

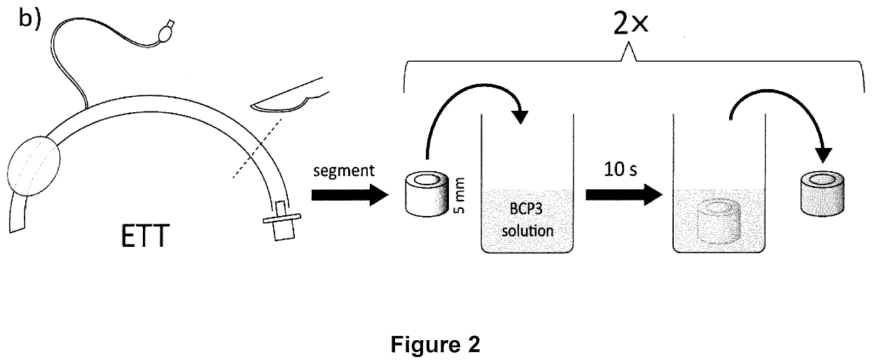

All ETT segments were dip-coated twice (FIG. 2). The coated segments are labelled X:Y where X is the concentration of PLGA in the coating solution as % w/v and Y is the concentration of BCP3 in the coating solution as mg/mL. It was observed that two coats resulted in a more consistent and uniform coating. Uncoated ETT segments and segments only coated with a 5% w/v PLGA solution were used as controls.

### Thickness Measurements

Coating thicknesses were determined via scanning electron microscopy (SEM) analysis of the tube segment cross-sections.

Thickness Measurement of BCP3-PLGA Coatings Via SEM:

Coated ETT segments were cut vertically using a fresh scalpel blade, mounted on aluminum stubs with double-sided carbon tabs, and coated with iridium (60 mA, 30 s) using a Cressington 208HRD sputter coater prior to imaging. Zeiss Merlin FESEM (Field Emission Scanning Electron Microscope) operated in the secondary electron (SE) mode to highlight topographical features with an accelerating voltage of 3 kV was used for imaging. Images of the ETT segment cross sections were obtained from each different BCP3: PLGA formulations. The acquired images were subsequently analyzed using ImageJ (NIH, USA) software to determine the thickness of coating present on each ETT segment surface, with thicknesses ranging from 4 to 20 μm for the various coating formulations used in this study.

A clear contrast could be observed between the PVC tube material and the PLGA-BCP3 coating, which allowed for the facile determination of coating thickness. The coating appeared to be deposited uniformly on the ETT surface, with the addition of BCP3 increasing the presence of particulates throughout the coating cross-section (FIG. 2).

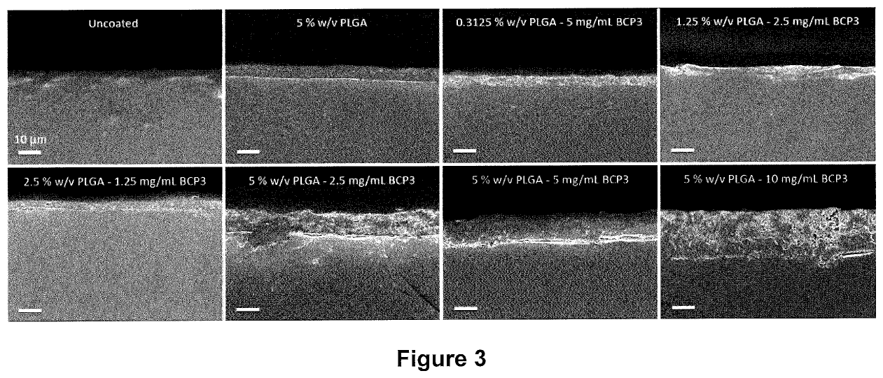

FIG. 3a demonstrates the effect of PLGA concentration on the coating thickness with increasing BCP3 concentration. The effect of BCP3 concentrations is more prominent at higher PLGA concentrations, whereby the thickness increases more significantly as the BCP3 concentration is increased. The PLGA effectively acts as a glue, allowing the BCP3 to be bound in the coating. Using higher PLGA concentrations, more BCP3 can be incorporated and held together by the polymer following the coating process.

### Determination of BCP3 Loading

BCP3 is minimally soluble in water, however in the presence of bases such as NaOH, BCP3 becomes highly soluble as a result of the deprotonation of the carboxylic acid groups. Furthermore, PLGA is a polyester polymer that is susceptible to base hydrolysis. Taking advantage of this, an extraction method with a NaOH solution was utilized to determine the BCP3 loading on the ETT segments.

Determination of BCP3 Loading Via NaOH Extraction:

Each of the twenty different dip-coated ETT segments were placed in 12 mL of 2.5 M NaOH solution in glass vials for 24 h to allow the BCP3 to dissolve and PLGA to degrade. From each vial, 100 μL of solution was transferred to a flat-bottom 96-well plate (Nunc) and was read using a BioTek plate reader at 339 nm. The concentration in each extracted solution was determined via comparison to a standard curve of BCP3 in 2.5 M NaOH in water. These concentrations were then used to determine the loadings as mg of BCP3 per cm² of ETT surface.

After 24 hours it was observed that the coating was completely removed from the ETT surface, while the BCP3 was completely dissolved. FIG. 3b summarizes the amount of BCP3 loading at the selected PLGA coating concentrations. It is possible to see a linear relationship between the concentration of PLGA and the concentration of BCP3 in the coating solution. As the PLGA concentration increases, the polymer provides better support and binding for the BCP3 thus leading to higher loading levels in the ETT coatings. The BCP3 loading also correlates very well with the thickness measurements, whereby the thickness of the coatings increase more significantly at higher PLGA concentrations, as the BCP3 concentration is increased during the coating process. It is also possible to see that by simply varying the BCP and PLGA ratios and concentrations, the BCP3 loading in the coating can be varied as desired.

### Example 2

### In Vitro Release of BCP3

PLGA-BCP3 coatings are designed as release platforms to allow the release of BCP3 to inhibit bacterial growth around the endotracheal tube when in use. BCP3 is a hydrophobic small molecule and PLGA is a hydrophobic copolymer, so it is important to determine if BCP3 can be sufficiently released from the coatings. The release profile of BCP3 may be expected to be initially via diffusion followed by a combination of diffusion and release via PLGA degradation owing to the degradable nature of PLGA as a result of the ester linkages present in the polymer backbone. To determine the release profile of BCP3 from the coatings, an in vitro release assay in PBS was conducted.

In Vitro BCP3 Release Study:

All formulations of coated ETT segments were cut vertically into equal quarters to produce curved rectangular pieces. Each quarter was placed into a well of tissue culture polystyrene (TOPS) 48-well plate. 500 μL of fresh 1×PBS was pipetted into each well containing the ETT quarters and the complete coverage of each piece with the PBS was ensured. The plate was sealed using parafilm and aluminum foil and placed in a shaker incubator at 37° C. (80 rpm). At 1, 4, 24 and 48 h time points, 100 μL of solution was removed and placed in a clear TOPS 96-well plate. The removed solution in each well was immediately replaced with 100 μL of fresh PBS at each time point. At 72 h and 8, 24 and 31 day time points all 500 μL of solution was removed, 100 μL was transferred to a TOPS 96-well plate and each well containing ETT segments was replaced with 500 μL of fresh PBS. The 96-well plates with the transferred solutions from each time point were read using a BioTek plate reader at 339 nm. The concentrations in each extracted solution was determined via comparison to a standard curve of BCP3 in 1×PBS. These concentrations were then used to determine the amount of BCP3 released at each time point as mg of BCP3 per cm2 of ETT surface per mL of PBS.

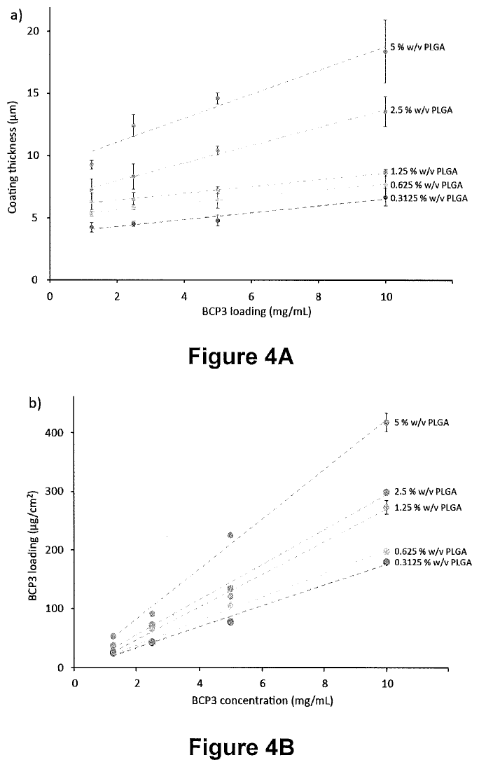

As seen in FIG. 4a, the release profiles for all twenty formulations of PLGA-BCP3 coatings for 31 days are presented. When the first 72 h is analyzed (FIG. 4b), significant release of BCP3 takes place within the first 24 h, with a range of 9-105 μg/ml·cm⁻² for the different coating formulations. The BCP3 and PLGA concentrations significantly affect the amount of BCP3 released in the first 24 h, with the higher BCP3 loadings leading to higher BCP3 release as expected. However the amount of BCP3 released plateaus from 24 to 48 h for all the formulations. In the first 24 h only 100 μL of incubated solution was removed for analysis to determine the release profile and maximum released amount, however at all time points after 48 h, all of the incubated solution was removed and replaced with fresh PBS to determine if the release of BCP3 was concentration dependent. As can be seen in FIG. 4b, even though the release rate of BCP3 had significantly reduced by 48 h, following replacement with fresh PBS, the release rate of BCP3 increases significantly, with a gradient similar to that of between 4 and 24 h. This increase was more substantial for coatings with higher BCP3 loadings as expected.

In FIG. 4a it is possible to see that especially for coatings with a higher BCP3 content, the total amount of released BCP3 continues to increase although the release rate decreases at each time point as the BCP3 in the coatings is depleted over time. While the release of BCP3 from the coatings with lower loadings plateaued at earlier time points, a significant measurable release could be observed for the higher loading coatings up to 31 days. Especially in the case of 5:10, 5:5, 2.5:10, and 1.25:10 coatings, the total amount of released BCP3 was still increasing after 31 days.

By varying the BCP3 to PLGA ratios and concentrations in the coating solutions, we have demonstrated that a range of BCP3 release profiles can be obtained. This allows for the preparation of coatings that can be fine-tuned to specific release profiles for various target applications.

### Example 3

Inhibition of S. aureus and P. aeruginosa Growth

S. aureus and P. aeruginosa are two major culprits associated with VAP. Various methods have been employed to reduce incidence of VAP including the most widely studied use of silver coated ETTs. In this study we utilized BCP3 as an active antimicrobial in PLGA-BCP3 coatings, and determined its activity towards gram positive S. aureus; both methicillin-sensitive (MSSA) and resistant (MRSA) strains and the Gram-negative P. aeruginosa.

Determination of the Minimum Inhibitory Concentration:

Three bacterial strains—two Gram-positive bacteria: Staphylococcus aureus (ATCC 29213) (MSSA) and methicillin resistant-S. aureus (MRSA) (ATCC 43300); and a Gram-negative Pseudomonas aeruginosa (ATCC 27853) were used in this study. Bacterial stocks (stored at −80° C. in nutrient broth with 15% glycerol) were streaked onto nutrient agar (Oxoid, Basingstoke, UK) plates for use as the working stock. From the stock an overnight bacterial culture grown in nutrient broth was diluted 1:100 into specific growth media, including tryptic soya broth for S. aureus and Luria-Bertani broth for P. aeruginosa. The final concentration of bacteria was made up to 1×10⁵ cfu/mL for the inhibition assays. BCP3 was suspended in respective bacterial media to afford 5 mg/mL suspension. The stock suspension was then twice and ten times diluted to finally obtain concentrations ranging from 8×10⁻⁴-5 mg/mL. Aliquots of 20 μL of each concentration were then added in quadruplicate to wells containing 80 μL of 1×10⁵ cfu/mL bacterial solutions in a 96 well plate format for final well concentrations of 1.6×10⁻⁴-1 mg/mL of BCP3. The plates were then sealed with parafilm and placed in a shaker incubator for 24 h at 37° C. After 24 h, 100 μL from each well was transferred into a TOPS 96 well plate (Nunc) and the optical density was measured at 600 nm using a BioTek plate reader. The inhibition of bacterial growth was determined relative to the control (%).

Table 51 summarizes the MIC values for MSSA, MRSA, and P. aeruginosa. For MSSA and MRSA an MIC of 15.6 μg/mL was observed. On the other hand, even at 1 mg/mL, a maximum of 32% inhibition of P. aeruginosa growth was observed, hence no MIC value was obtained for P. aeruginosa at these concentrations. This is not unexpected since gram-negative bacteria including P. aeruginosa possess lipopolysaccharide containing outer membrane which can act as a barrier to significantly reduce the effects of antimicrobials, hence the interaction of BCP3 with the MscL channel could also be reduced as a result.

TABLE S1
BCP3 MIC* values for MSSA, MRSA and P. aeruginosa.

| Bacterial strain | MIC (μg/mL) |
|---|---|
| MRSA | 15.6 |
| MSSA | 15.6 |
| P. aeruginosa | >1000 |

*lowest concentration at which greater than 90% inhibition of bacterial growth was observed.

As demonstrated by the MIC study, BCP3 in its free from in solution is able to significantly inhibit the growth of MSSA and MRSA and reduce the growth of P. aeruginosa. From our in vitro studies, BCP3 could be released at a range of concentrations from the PLGA-BCP3 coatings, reaching concentrations above the MICs for MSSA and MRSA.

It is important to determine if the released BCP3 from the coatings can also inhibit bacterial growth. In order to investigate this, an in vitro study was conducted whereby ETT segments coated with various PLGA-BCP3 formulations, were placed in MSSA, MRSA and P. aeruginosa cultures for 24 h. Subsequently the growth of these bacteria were determined relative to uncoated and only PLGA coated ETT segments.

Inhibition of S. aureus and P. aeruginosa Growth in the Presence of BCP3-PLGA coatings:

Three bacterial strains—gram positive Staphylococcus aureus (ATCC 29213) (MSSA), methicillin resistant-Staphylococcus aureus (MRSA) (ATCC 43300) and gram negative Pseudomonas aeruginosa (ATCC 27853) were used in this study. Bacterial stocks (stored at −80° C. in nutrient broth with 15% glycerol) were streaked onto nutrient agar (Oxoid, Basingstoke, UK) plates for use as the working stock. From the stock an overnight bacterial culture grown in nutrient broth was diluted 1:100 into specific growth media, including tryptic soya broth for S. aureus and Luria-Bertani broth for P. aeruginosa. The final concentration of bacteria was made up to 1×10⁵ cfu/mL for the inhibition assays. All formulations of coated ETT segments were cut vertically into equal quarters to produce curved rectangular pieces. Each quarter was placed into a well of tissue culture polystyrene (TOPS) 48-well plate in quadruplicates. From each bacterial solution, 500 μL was then added to wells containing the coated ETTs. PLGA coated and uncoated ETT segments were used as controls. The plates were then sealed with parafilm and placed in a shaker incubator for 24 h at 37° C. After 24 h, 100 μL from each well was transferred into a TOPS 96 well plate (Nunc) and the optical density was measured at 600 nm using a BioTek plate reader. The inhibition of bacterial growth was determined relative to the control (%).

FIG. 5 summarizes bacterial growth inhibition for various PLGA-BCP3 formulations after 24 h. For both MSSA and MRSA, a significant growth inhibition can be observed for all of the coating formulations. For MSSA, a concentration dependent inhibition can be observed for each PLGA concentration group, whereby a decrease in inhibition is observed as the BCP3 concentration is reduced (FIG. 5a). Furthermore the highest inhibition is observed for 5 and 2.5% w/v PLGA, and as the PLGA concentration is reduced the inhibition to MSSA growth is also reduced for 1.25, 0.625 and 0.3125% w/v. Coatings 5:10, 5:5, 2.5:10, 2.5:5 and 1.25:10 were able to inhibit growth by >95%, and even at the lowest BCP3 loading (0.3125:10) a significant reduction in MSSA growth (>60% inhibition) can still be observed.

For MRSA, a significant inhibition in growth is observed with an average of 80% reduction across all the coating formulations (FIG. 5b). However, the maximum reduction observed was 91% at the highest PLGA and BCP3 loading (5:10), on the other hand >95% reduction was observed for MSSA at various formulations. Furthermore, a strong concentration dependent inhibition is not apparent for MRSA at different PLGA concentrations, with the inhibition being relatively constant across all coating formulations.

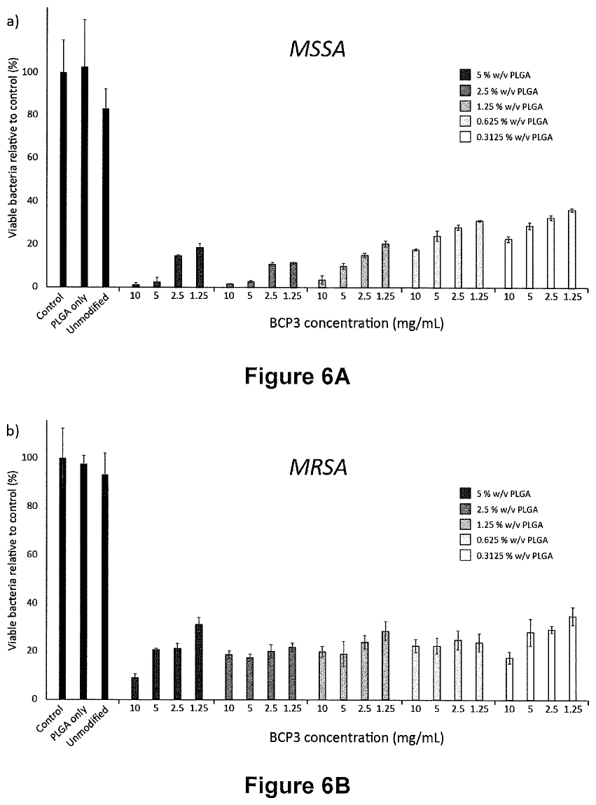

The MIC study demonstrated that P. aeruginosa was not as sensitive to BCP3 as were MSSA and MRSA, however some growth inhibition was observed. P. aeruginosa was also exposed to coated ETT segments for 24 h. The inhibition of P. aeruginosa growth was to a lower extent compared to MSSA and MRSA (FIG. 6). At 5, 2.5 and 1.25% w/v PLGA concentrations an average of 50% reduction in P. aeruginosa growth was observed, with a maximum of 63% reduction at 1.25:10 coatings.

MIC assays demonstrated that BCP3 possessed excellent growth inhibition properties towards both MSSA and MRSA, while still being able to reduce the growth of P. aeruginosa. Furthermore the PLGA-BCP3 coatings allowed the release of BCP3, such that in all formulations of the coating, the growth of MSSA and MRSA was significantly inhibited. Although to a lower extent, a significant inhibition of P. aeruginosa growth was also observed in the presence of the coated ETTs.

### Example 4

### In Vitro Cytotoxicity Studies

An ideal antimicrobial compound should be effective in inhibiting bacterial growth while remaining non-cytotoxic towards the host. While silver, which has been studied and utilized for ETTs,^{[23]} is effective against a range of bacteria, its cytotoxic nature towards mammalian cells is of concern. To determine whether BCP3 possesses cytotoxic properties, an in vitro cell viability assay was conducted at low and high concentrations of BCP3.

Determination of Minimal BCP3 Cytotoxic Concentration:

BCP3 was sterilized via incubation in 80% ethanol in a sterile glass vial for 1 h prior to being vacuum dried at 120° C. for 5 h (0.2 mbar). Subsequently BCP3 was suspended in complete minimum essential medium (MEM, containing 10% (v/v) FBS and 1% (v/v) non-essential amino acids) to afford a 5 mg/mL suspension. The stock suspension was then twice and ten times diluted to finally obtain concentrations ranging from 8×10⁻⁴-5 mg/mL. Aliquots of 20 μL of each concentration were then added in quadruplicate to wells previously seeded with L929 cells containing 80 μL of complete MEM (18,000 cells per well, 16 h) in a 96 well plate format for final well concentrations of 1.6×10⁻⁴-1 mg/mL. Additional controls were added to wells containing pre-seeded cells; medium alone, 5%, and 10% (v/v) dimethylsulfoxide (DMSO, Sigma) in medium. The plate was subsequently incubated for 21 h (37° C., 5% CO₂). An 3-(4,5-dimethylthiazol-2-yl)-5(3-carboxymethoxyphenyl)-2-(4-sulfophenyl)-2H-tetrazolium (MTS) assay was then utilized to determine cell viability based on metabolic activity of cells. Stock solutions of 4 mM MTS (Promega) in PBS and 3 mM of phenazine methosulfate (PMS, Sigma) in PBS were used to make up a mixture of MTS and PMS reagent. A working assay reagent solution was made up by addition of 2 mL of MTS and 100 μL of PMS stock solutions per 10 mL of complete medium. Media was removed from the sample and control wells and washed with 120 μL of fresh medium; subsequently 100 μL of the assay reagent solution was added to wells then incubated for 3 h at 37° C., 5% CO₂. For colorimetric analysis, plates were read in a BioTek plate reader at wavelengths 490 nm and 655 nm. The difference in readings at the two wavelengths was used to calculate cell attachment compared to controls.

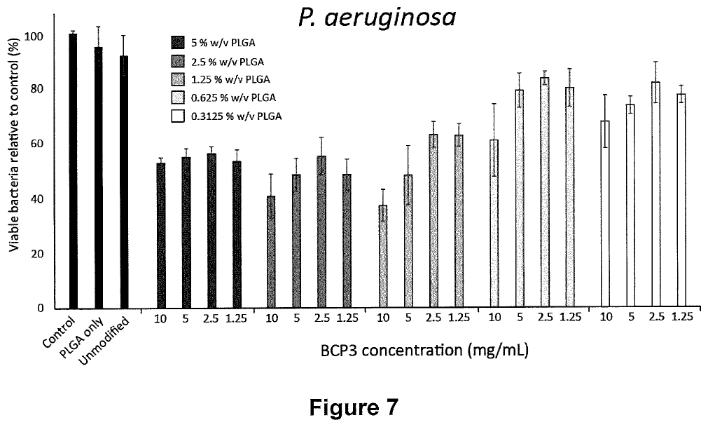

At all concentrations of BCP3, even at 1 mg/mL, no cytotoxicity was observed (FIG. 7a). A minor reduction in cell viability compared to a TOPS control was observed with increasing concentrations, however at all concentrations the L929 cell viability was above 70% (85% at 1 mg/mL), hence BCP3 was considered non-cytotoxic.

BCP3 in the free form showed non-cytotoxic properties with L929 fibroblasts. However, for the preparation of PLGA-BCP3 coatings, the organic solvent THF is used. PLGA is a widely studied polymer which is approved by the FDA for a variety of applications and is considered non-toxic. Nevertheless, it is important to determine if the coating process and the use of an organic solvent can lead to the leaching of potentially cytotoxic compounds from the coating.

It was further necessary to determine if any cytotoxic leachables were present in the PLGA-BCP3 coatings.

In Vitro Cytotoxicity Studies of BCP3-PLGA Coating:

Cytotoxicity assessment of coated ETTs was performed according to the International Standard 15010993-5/12. All formulations of coated ETT segments were cut vertically into equal quarters to produce curved rectangular pieces. Each quarter was placed into a well of tissue culture polystyrene (TOPS) 48-well plate. To each well containing ETT segments, 500 μL of complete minimum essential medium (MEM, containing 10% (v/v) FBS and 1% (v/v) non-essential amino acids) was added and the plate was allowed to incubate for 66 hours in a sealed humidified chamber placed on a rocker (Seoulin Mylab™) at 20 rpm at 37° C. in a 5% CO₂ incubator. PLGA coated and uncoated ETT segments were used as controls.

Aliquots of 100 μL of incubated medium from each well were added in quadruplicate to wells previously seeded with L929 cells (18,000 cells per well, 16 h) in a 96 well plate format. Additional controls were added to wells containing pre-seeded cells; medium alone, 5%, and 10% (v/v) dimethylsulfoxide (DMSO, Sigma) in medium. The plate was subsequently incubated for 21 h (37° C., 5% CO₂). An MTS assay was performed to assess cell viability as previously described for determination of BCP3 cytotoxic concentration.

No cytotoxicity was observed for any of the coating formulations (FIG. 7b), even at the highest BCP3 loadings. BCP3 and the PLGA-BCP3 coatings show no cytotoxicity towards L929 fibroblasts, and combined with the strong inhibitory properties of BCP3 towards MSSA and MRSA, and even towards P. aeruginosa, the coating platform described in this study possesses highly desirable properties to reduce infections associated with ETT use.

### Example 5

### Dip-Coating of Entire ETT

For the scale up of a coating on a medical device, it is necessary for the coating process to be simple, adaptable and translatable for large scale production. Dip-coating is a robust and simple coating method that is widely utilized for a variety of medical and non-medical applications, as reported in A. Simchi, E. Tamjid, F. Pishbin, A. R. Boccaccini, “Recent progress in inorganic and composite coatings with bactericidal capability for orthopaedic applications”, Nanomedicine: Nanotechnology, Biology and Medicine 2011, 7, pp 22-39; and in E. M. Hetrick, M. H. Schoenfisch, “Reducing Implant-Related Infections: Active Release Strategies”, Chemical Society Reviews 2006, 35, 780, the disclosures of which are hereby incorporated in their entirety. In this study we have demonstrated that small ETT segments can be easily coated by placing the segments into a PLGA-BCP3 solution, however it is important to demonstrate if this coating procedure can be applied to the entire ETT, as would be required for real-world applications. To demonstrate this, an entire Medline endotracheal tube was coated using a simple dip-coating procedure.

Coating of an Entire ETT Using a Dip-Coating Method:

For coating of an entire ETT, a Medline 7.5 mm internal diameter poly(vinyl chloride) (PVC) endotracheal tube (ETT) (Medline Industries, IL, USA) was utilized. The balloon section of the tube was wrapped and sealed with polytetrafluoroethylene (PTFE) tape to avoid swelling and damage to the balloon. A solution of 80 mL of 1.25 mg/mL BCP3, 1.25% w/v PLGA was prepared and sonicated for 20 min before being transferred into a 2 cm ID, 40 cm length glass cylinder. For the dip-coating procedure, the ETT was held using a clean glass rod (8 mm diameter) by inserting the tip of the glass rod into the balloon end of the ETT. The ETT was then introduced into the cylinder with the coating solution and removed in a total of 10 seconds (see Supplementary Video). Subsequently the ETT was hung vertically and allowed to dry for 5 min before repeating the dip coating procedure. The ETT was then allowed to dry in a laminar flow hood for 1 h before inspection and photographing. The PTFE tape was removed to expose the balloon section and a syringe was used to inflate the balloon to ensure function.

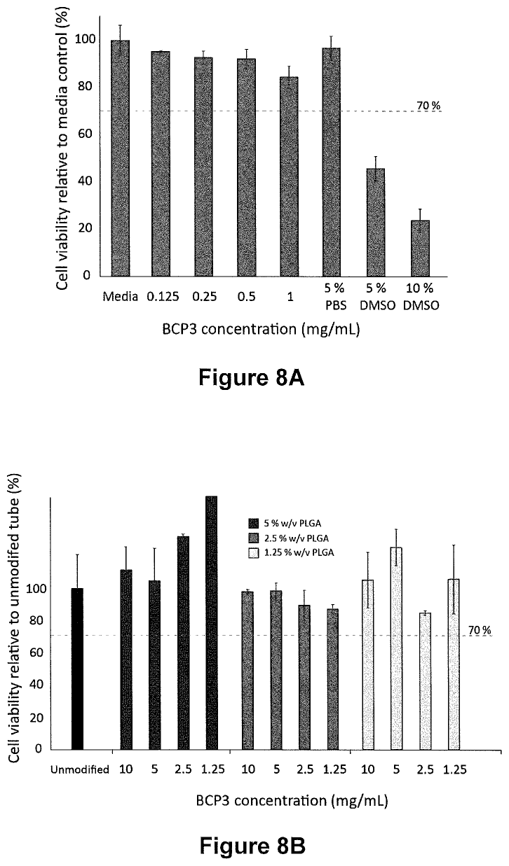

Observations on the ETT indicated a visually uniform coating throughout the tube both on the internal and outside surface (FIG. 8b). No streaks or patches were observed on the surface. Importantly, all of the manufacturer's printed features including branding and depth indicators were unaffected by the dip coating process (FIG. 8c). The balloon segment of the ETT was also unaffected by the dip coating process and could be repeatedly inflated and deflated with a syringe.

Coating of the entire ETT was successfully achieved using a simple dip-coating process. We have demonstrated that uniform coating with PLGA-BCP3 in THF could easily be scaled up from coating 5 mm segments to entire ETTs, allowing coating both inside and outside at the same time. The ease of coating application using the simple dip-coating process, combined with the commercial availability of PLGA, make these coatings highly amenable for large scale manufacturing and application.

### Synthesis, Antimicrobial Activity & Cytotoxicity, of Compounds of Formula (I)

The Synthesis, Antimicrobial Activity and Cytotoxicity, of Compounds of Formula (I) have been previously examined and published in US 2015/0189873 and in Chem. Eur. J. 2013, 19, 17980-17988, the disclosures of both of which are incorporated herein in their entirety.

### Lipophillicity and Solubility of Compounds of Formula (I)

The general applicability of antimicrobial compounds of Formula (I) to the coatings of the invention comprising biodegradable polymers, is understood to be influenced by the in vivo physical interactions between the antimicrobial compounds and the biodegradable polymer matrix. Antimicrobial compounds of Formula (I) are characterised by having relatively high octanol:water partition coefficient (LogP) values and relatively low aqueous solubility (LogS) values.

For example, the LogP and Log S values of antimicrobial compounds of Formulae A to E were calculated using ALOGPS 2.1, an algorithm accessible at: http://www.vcclab.org/lab/alogps/, the results are presented below in Table 2.

TABLE 2
Partition Coefficients and Solubility of Compounds of Formula (I)

| Compound | LogP | LogS |
|---|---|---|
| Formula A | 5.46 | −7.10 |
| Formula B | 4.97 | −7.41 |
| Formula C | 5.28 | −7.30 |
| Formula D | 5.45 | −7.10 |
| Formula E | 5.64 | −7.33 |

These LogP and LogS values suggest that the antimicrobial compounds of Formula (I) are highly lipophilic and of very low solubility in aqueous biological fluids and that they are therefore not capable of crossing membranes as a result of their high affinity to the lipid membrane. As a result, an equilibrium between the amount of antimicrobial compound released from the polymer and that present in the vicinity of the coating or device will be reached sooner translating to an even longer lasting release profile in vivo. The hydrogen bonding and partitioning interactions between biodegradable polymers such as PLGA and the compounds of Formula (I), such as the compound of Formula E, play an important role in the release of compounds of Formula (I) from the polymer. On one hand there is the release of the compounds of Formula (I) from a high concentration in the polymer to a low concentration in the environment due to diffusion. On the other hand an opposing force is the H-bonding between biodegradable polymers such as PLGA and the compounds of Formula (I) pulling back on the compound of Formula (I) and thereby resisting its release due to diffusion. A further interaction contributing to the controlled release of antimicrobial compounds of Formula (I) from biodegradable polymers such as PLGA is the tendency of such polymers to form internal hydrophobic pockets favourable to the retention of compounds of Formula (I) due to the generally high lipophilicity of compounds of Formula (I). On this basis, it is to be understood that similarly advantageous results will be observed from coatings comprising compounds of Formula (I) with other biodegradable polymers having a similar hydrogen-bonding and hydrophobic pocket forming capacity, such as polylactide, polyglycolide, polyester, polyurethane and combinations thereof, and combinations of PLGA with polylactide, polyglycolide, polyester, polyurethane.

1. A composition for coating a surface, said composition comprising;
a. a polymer selected from the group: poly(lactic-co-glycolic acid), polylactide, polyglycolide, polyester, polyurethane and combinations thereof; and
b. an antimicrobial compound of Formula (I);
wherein the compound of Formula (I) is a compound having a structure selected from Group I, wherein Group I consists of; 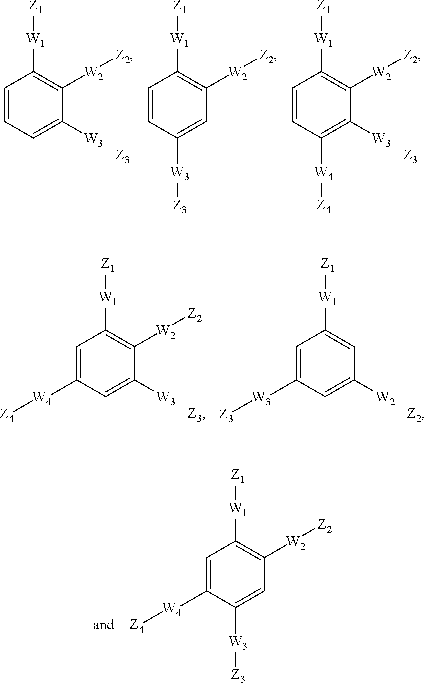
wherein
each of W₁, W₂, W₃, and W₄ is the same and is selected from the group consisting of C₂₋₄ alkyl, substituted C₂₋₄ alkyl; and C₂ alkene;
each of Z₁, Z₂, Z₃, and Z₄ is the same and each is selected from the group consisting of; 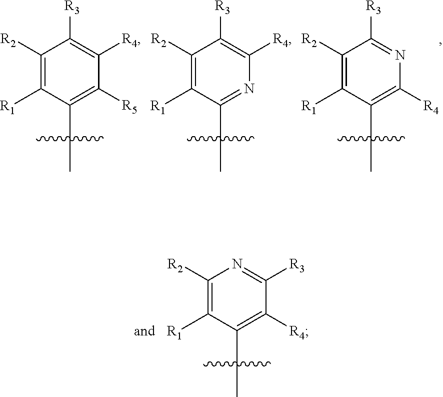
each of R₁, R₂, R₃, R₄, and R₅ is independently H or C₁₋₈ heteroalkyl, wherein the C₁₋₈ heteroalkyl comprises —CO₂H or an ester thereof, with the proviso that at least one of R₁, R₂, R₃, R₄, and R₅ is C₁₋₈ heteroalkyl, or a pharmaceutically acceptable salt thereof.

2. The composition for coating a surface of claim 1 wherein the polymer is a biodegradable polymer.

3. The composition for coating a surface according to claim 1 or claim 2 wherein the C₁ heteroalkyl group of the compound of Formula (I) is —CO₂H or an ester thereof.

4. A composition for coating a surface according to any one of claims 1 to 3 characterised in that the polymer is poly(lactic-co-glycolic acid).

5. A composition according to any one of claims 1 to 4 characterised in that the composition provides a concentration dependent release of an antibiotic compound of Formula (I).

6. The composition according to claim 5, characterised in that the concentration dependent release of an antibiotic compound of Formula (I) is a self-limiting drug release profile.

7. A composition for coating a surface, wherein the composition comprises;
a. a polymer selected from the group: poly(lactic-co-glycolic acid), polylactide, polyglycolide, polyester, polyurethane and combinations thereof; and
b. an antimicrobial compound of Formula (I) as defined in claim 1;
wherein the concentration of the antimicrobial compound of Formula (I) in the composition is at least 0.01 mg/mL.

8. A composition according to claim 7 characterised in that the polymer is poly(lactic-co-glycolic acid).

9. A composition according to claim 7 or claim 8 characterised in that the polymer is a biodegradable polymer.

10. A coating, wherein the coating comprises;
a. a polymer selected from the group: poly(lactic-co-glycolic acid), polylactide, polyglycolide, polyester, polyurethane and combinations thereof; and
b. an antimicrobial compound of Formula (I) as defined in claim 1;
characterised in that the amount of the antimicrobial compound of Formula (I) per unit surface of the coating is at least 0.2 μg/cm2.

11. A coating according to claim 10 characterised in that the polymer is poly(lactic-co-glycolic acid).

12. A coating according to claim 10 or claim 11 characterised in that the polymer is a biodegradable polymer.

13. A coating, wherein the coating comprises;
a. a polymer selected from the group: poly(lactic-co-glycolic acid), polylactide, polyglycolide, polyester, polyurethane and combinations thereof; and
b. an antimicrobial compound of Formula (I) as defined in claim 1;
and wherein, when a sample of the coating is placed into a well of tissue culture polystyrene (TOPS) 48-well plate and 500 μL of fresh 1× phosphate-buffered saline is pipetted into each well containing the sample of coating such that complete coverage of each sample of the coating with the phosphate-buffered saline is ensured, and the well of tissue culture polystyrene is sealed and placed in a shaker incubator at 37° C. (80 rpm), the concentration of the antimicrobial compound of formula (I) in the solution at 24 hours is at least 0.05 μg of the compound of Formula (I) per cm2 of coating surface per mL.

14. A coating according to claim 13 characterised in that the polymer is poly(lactic-co-glycolic acid).

15. A coating according to claim 13 or claim 14 characterised in that the polymer is a biodegradable polymer.

16. A coating according to any one of claims 10 to 15 characterised in that the concentration of the antimicrobial compound of Formula (I) at the surface to which the coating is applied is an effective or useful inhibitory concentration for bacteria selected from the group: gram positive bacteria or gram negative bacteria or combinations thereof.

17. A coating according to any one of claims 10 to 16 characterised in that the concentration of the antimicrobial compound of Formula (I) at the surface to which the coating is applied is an effective or useful inhibitory concentration for bacteria selected from the group: S. aureus or P. aeruginosa or combinations thereof.

18. A coating according to any one of claims 10 to 17, wherein the coating comprises;
a. a polymer selected from the group: poly(lactic-co-glycolic acid), polylactide, polyglycolide, polyester, polyurethane and combinations thereof; and
b. an antimicrobial compound of Formula (I) as defined in claim 1;
wherein, when the coating is placed in an aqueous solution, and wherein, when a sample of the coating is placed into a well of tissue culture polystyrene (TOPS) 48-well plate and 500 μL of fresh 1× phosphate-buffered saline is pipetted into each well containing the sample of coating such that complete coverage of each sample of the coating with the phosphate-buffered saline is ensured, and the well of tissue culture polystyrene is sealed and placed in a shaker incubator at 37° C. (80 rpm), and wherein at 1, 4, 24 and 48 h, 100 μL of solution was removed, and wherein at 72 h and at 8 and 24 days all 500 μL of solution was removed and replaced with 500 μL of fresh 1× phosphate-buffered saline, the concentration of antimicrobial compound in the aqueous solution after 31 days is at least 5 μg of the compound of Formula (I) per cm² of coating surface per mL.

19. A coating according to any one of claims 10 to 18, wherein the coating comprises:
the thickness of the coating is at least 4 μm and the concentration of the compound of Formula I is at least 25 μg/cm².

20. A coating or composition according to any one of the preceding claims characterised in that the coating is applied to a surface forming part of a medical device.

21. A coating or composition according to claim 20 characterised in that the medical device is an endotracheal tube.

22. A coating or composition according to any one of the preceding claims characterised in that the antimicrobial compound of Formula (I) has a LogP value of 5.4±1.5.

23. A coating or composition according to any one of the preceding claims characterised in that the antimicrobial compound of Formula (I) has a LogP value of 5.4±1.0.

24. A coating or composition according to any one of the preceding claims characterised in that the antimicrobial compound of Formula (I) has a LogP value of 5.4±0.54.

25. A coating or composition according to any one of the preceding claims characterised in that the antimicrobial compound of Formula (I) has a LogS value of −7.3±1.0.

26. A coating or composition according to any one of the preceding claims characterised in that the antimicrobial compound of Formula (I) has a LogS value of −7.3±0.5.

27. A coating or composition according to any one of the preceding claims characterised in that the antimicrobial compound of Formula (I) has a LogS value of −7.3±0.3.

28. A coating or composition according to any one of the preceding claims characterised in that the antimicrobial compound of Formula (I) has a LogS value of −7.3±0.2

29. A coating or composition according to any one of the preceding claims characterised in that the antimicrobial compound of Formula (I) has a LogS value of −7.25±0.16.

30. A coating or composition according to any one of the preceding claims characterised in that the antimicrobial compound of Formula (I) has a LogP value of 5.4±0.5 and a LogS value of −7.3±0.2.

31. A coating or composition according to any one of the preceding claims characterised in that the antimicrobial compound of Formula (I) is a compound of Formula A, or an ester or pharmaceutically acceptable salt thereof.

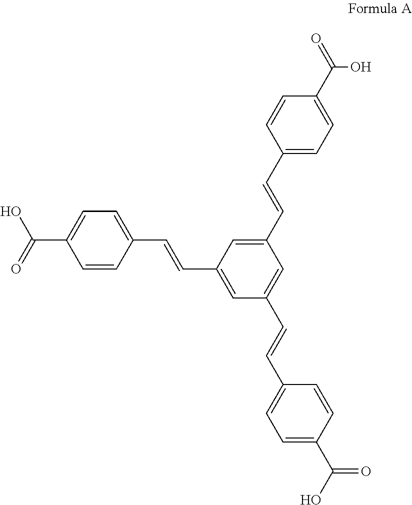 [SMILES: O=C(O)c1ccc(/C=C/c2cc(/C=C/c3ccc(C(=O)O)cc3)cc(/C=C/c3ccc(C(=O)O)cc3)c2)cc1]

32. A coating or composition according to any one of the preceding claims characterised in that the antimicrobial compound of Formula (I) is a compound of Formula B, or an ester or pharmaceutically acceptable salt thereof.

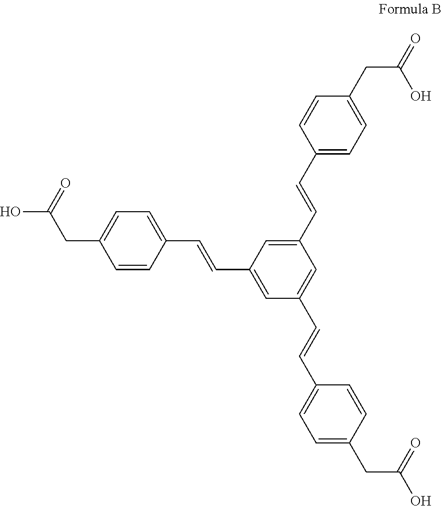 [SMILES: O=C(O)Cc1ccc(/C=C/c2cc(/C=C/c3ccc(CC(=O)O)cc3)cc(/C=C/c3ccc(CC(=O)O)cc3)c2)cc1]

33. A coating or composition according to any one of the preceding claims characterised in that the antimicrobial compound of Formula (I) is a compound of Formula C, or an ester or pharmaceutically acceptable salt thereof.

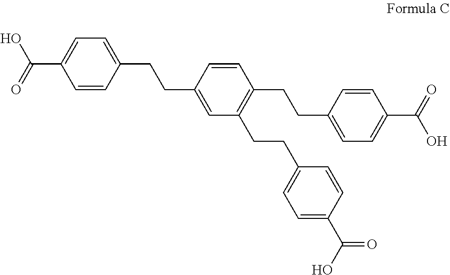 [SMILES: O=C(O)c1ccc(CCc2ccc(CCc3ccc(C(=O)O)cc3)c(CCc3ccc(C(=O)O)cc3)c2)cc1]

34. A coating or composition according to any one of the preceding claims characterised in that the antimicrobial compound of Formula (I) is a compound of Formula D, or an ester or pharmaceutically acceptable salt thereof.

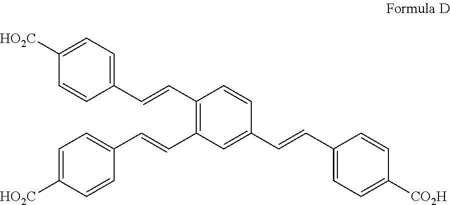 [SMILES: O=C(O)c1ccc(/C=C/c2ccc(/C=C/c3ccc(C(=O)O)cc3)c(/C=C/c3ccc(C(=O)O)cc3)c2)cc1]

35. A coating or composition according to any one of the preceding claims characterised in that the antimicrobial compound of Formula (I) is a compound of Formula E, or an ester or pharmaceutically acceptable salt thereof. 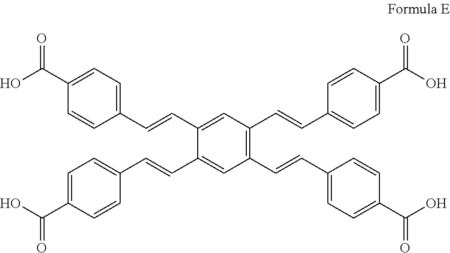 [SMILES: O=C(O)c1ccc(/C=C/c2cc(/C=C/c3ccc(C(=O)O)cc3)c(/C=C/c3ccc(C(=O)O)cc3)cc2/C=C/c2ccc(C(=O)O)cc2)cc1]
